(19) United States
(12) Patent Application Publication
(10) Pub. No.: US 2013/0158265 A1
(43) Pub. Date: Jun. 20, 2013

(54) SITAGLIPTIN, SALTS AND POLYMORPHS THEREOF

(71) Applicants: Dhananjay Govind Sathe, Govandi Mumbai (IN); Subhash Vishwanath Damle, Mambai (IN); Nitin Dnyaneshwar Arote, Govandi Mumbai (IN); Rakesh Ramchandra Ambre, Govandi, Mumbai (IN); Kamlesh Digambar Want, Govandi, Mumbai (IN); Tushar Anil Naik, Mumbai (IN)

(72) Inventors: Dhananjay Govind Sathe, Govandi Mumbai (IN); Subhash Vishwanath Damle, Mambai (IN); Nitin Dnyaneshwar Arote, Govandi Mumbai (IN); Rakesh Ramchandra Ambre, Govandi, Mumbai (IN); Kamlesh Digambar Want, Govandi, Mumbai (IN); Tushar Anil Naik, Mumbai (IN)

(21) Appl. No.: 13/819,252

(22) PCT Filed: Aug. 26, 2011
(86) PCT No.: PCT/IN2011/000582

§ 371 Date: Feb. 26, 2013

(30) Foreign Application Priority Data
Aug. 27, 2010 (IN) 2396/MUM/2010

(51) Int. Cl. C07D 487/04

(52) U.S. Cl. CPC C07D 487/04
USPC 544/350

(57) ABSTRACT

The present invention relates to an improved process for preparation of Sitagliptin or pharmaceutically acceptable salts thereof. The present invention further relates to novel polymorphs of Sitagliptin salts and process for preparation thereof.

### RELATED APPLICATION

This application claims benefit of Indian Provisional Application No. 2396/MUM/2010 filed on 27^{th} Aug. 2010.

### FIELD OF THE INVENTION

The present invention relates to an improved process for preparation of Sitagliptin or pharmaceutically acceptable salts thereof. The present invention further relates to novel polymorphs of Sitagliptin salts and process for preparation thereof.

### BACKGROUND OF THE INVENTION

Sitagliptin phosphate is described chemically as 7-[(3R)-3-amino-l-oxo-4-(2,4,5-tri-fluorophenyl)butyl]-5,6,7,8-tetrahydro-3-(trifluoromethyl)-1,2,4-triazolo[4,3-a]pyrazine phosphate(1:1) and the structural formula is:

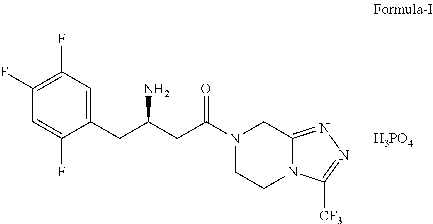

Sitagliptin phosphate is currently marketed in the United States under the trade name JANUVIA™. It is indicated to improve glycemic control in patients with type 2 diabetes mellitus. Sitagliptin is a DPP-4 inhibitor, which is believed to exert its actions in patients with type-2 diabetes by slowing the inactivation of incretin hormones.

U.S. Pat. No. 6,699,871 describes a general class of inhibitors of dipeptidyl peptidase-IV including Sitagliptin and pharmaceutically acceptable salts thereof. Sitagliptin hydrochloride is prepared from 2,4,5-trifluorobenzyl chloride. The major disadvantages of this process is that it involves multi-step synthesis; use of hazardous chemicals such as butyl lithium, diazomethane and silver benzoate; and low overall yield (18%).

WO2004087650 discloses a process for preparation of Sitagliptin phosphate which involves preparation of β-ketoester compound by combining 2,4,5-tri-fluorophenylacetic acid with monomethyl malonate potassium salt in presence of 1,1′-carbonyldiimidazole (CDI). The obtained β-ketoester is treated with an enantioselective catalyst in presence of hydrogen followed by treating with an aqueous base to obtain the corresponding hydroxyacid. The obtained hydroxyacid is converted to lactam compound by combining hydroxyacid with benzyl hydroxylamine in the presence of a coupling reagent followed by cyclocondensing with an azodicarboxylate in presence of a phosphine ligand. The obtained lactam is converted to protected Sitagliptin by treating with an aqueous base followed by treatment with triazole hydrochloride compound in presence of a coupling reagent. The protected Sitagliptin is subjected to debenzyloxylation in presence of palladium followed by phosphoric acid treatment to get Sitagliptin phosphate. This process leads to formation of multiple impurities thus not feasible on an industrial scale.

WO2004085661 describes a process for the preparation of enantiomerically enriched Sitagliptin via (S)-phenylglycine amide protected triazole intermediate, followed by hydrogenation in presence of platinum catalyst. The protected Sitagliptin intermediate is then de-protected in presence of palladium hydroxide to obtain Sitagliptin base. The main disadvantage of this process is that the chiral and the chemical purity of obtained Sitagliptin is less due to formation of desfluorinated impurities [more than 0.15% (not complying with ICH requirement)].

WO2006081151 describes a process for preparation of Sitagliptin via enamine compound which is reduced by employing a rhodium metal precursor complexed to a ferrocenyl diphosphine ligand, followed by treatment with phosphoric acid to obtain Sitagliptin phosphate.

WO2009084024 discloses a process for the preparation of (R)-Sitagliptin via enamine intermediate. The racemic Sitagliptin obtained is resolved using a chiral acid to get the desired isomer which is further converted to its phosphate salts. The disadvantage of this process is low overall yield (˜7%) as the process employs resolution of the racemic Sitagliptin base.

WO2010032264 describes an improved process for the preparation of Sitagliptin and its salt. The process involves the reduction of protected or unprotected prochiral β-aminoacrylic acid or derivative thereof, by using borane containing reducing agents at atmospheric pressure. The resulting racemic β-amino compound is resolved to a pure stereoisomer of Sitagliptin.

WO2005072530 discloses crystalline hydrochloric acid, benzenesulfonic acid, p-toluenesulfonic acid, 10-camphorsulfonic acid and tartaric acid salt of Sitagliptin.

WO2005003135 discloses Sitagliptin dihydrogen phosphate and its crystalline monohydrate. Four crystalline polymorphs of Sitagliptin dihydrogen phosphate anhydrate are disclosed in WO2005020920 and WO2005030127 .

WO2009085990 discloses crystalline hydrobromide, methane sulfonate, acetate, benzoate, oxalate, succinate, mandelate, fumarate, lactate and anhydrate dihydrogen phosphate salt of Sitagliptin.

WO201000469 discloses crystalline hydrochloride, fumarate, malate, sulfate, phosphate, succinate, lactate, glycolate, maleate, citrate, mesylate salt of sitagliptin.

WO2010012781 discloses novel crystalline forms of galactarate, hemi-L-malate, D-gluconate, sucinate, hydrobromide, thiocyanate, oxalate, L-asparate, ethanedisulfonate, pyroglutamate, glutarate, acetate forms of sitagliptin.

There exists a need for an improved process for preparation of Sitagliptin and pharmaceutically acceptable salts thereof that meets the ICH requirements. The present invention provides simple, industrially feasible and commercially viable process for the preparation of Sitagliptin or salt thereof with purity of more than 99.8%. The present invention further provides novel polymorphic forms of Sitagliptin salts and process for preparation thereof.

### OBJECT OF THE INVENTION

An object of the present invention is to provide a simple and industrially feasible process for preparation of Sitagliptin or pharmaceutically acceptable salts thereof. Another object of the present invention is to provide Sitagliptin or pharmaceutically acceptable salts thereof having genotoxic impurities below 100 ppm.

Another object of the present invention is to provide isolated compound selected from 3(R)-3-amino-1-[3-(trifluoromethyl)-5H,6H,7H,8H-[1,2,4]triazolo[4,3-a]pyrazin-7-yl]-4-(2,5-difluorophenyl)butan-1-one; or 3(R)-3-amino-1-[3-(trifluoro methyl)-5H,6H,7H,8H-[1,2,4]triazolo[4,3-a]pyrazin-7-yl]-4-(2,4-difluoro phenyl) butan-1-one; or 3(R)-3-amino-1-[3-(trifluoromethyl)-5H,6H,7H,8H-[1,2,4]triazolo[4,3-a]pyrazin-7-yl]-4-(3,4-difluorophenyl)butan-1-one phosphate and use thereof as reference marker/standard.

Another object of the present invention is to provide novel polymorphs of Sitagliptin HCl and process for preparation thereof.

Yet another object of the present invention is to provide novel polymorphs of Sitagliptin esylate and process for preparation thereof.

### BRIEF DESCRIPTION OF THE DRAWINGS

FIG. 1: X-ray diffraction pattern of Sitagliptin obtained according to the present invention.

FIG. 2: X-ray diffraction pattern of Sitagliptin phosphate obtained according to example 7.

FIG. 3: X-ray diffraction pattern of Sitagliptin phosphate obtained according to example 8.

FIG. 4: X-ray diffraction pattern of Sitagliptin phosphate monohydrate obtained according to the present invention.

FIG. 5: X-ray diffraction pattern of Sitagliptin hydrochloride Form III.

FIG. 6: X-ray diffraction pattern of Sitagliptin hydrochloride Form IV.

FIG. 7: X-ray diffraction pattern of Sitagliptin hydrochloride Form V.

FIG. 8: X-ray diffraction pattern of Sitagliptin Esylate Form I.

FIG. 9: X-ray diffraction pattern of Sitagliptin Esylate Form II.

FIG. 10: X-ray diffraction pattern of Sitagliptin Esylate Form III.

### SUMMARY OF THE INVENTION

According to one aspect of the present invention, there is provided a process for preparation of Sitagliptin or pharmaceutically acceptable salts thereof comprising the steps of,
a) hydrogenating methyl 4-(2,4,5-trifluorophenyl)-3-oxobutanoate in presence of less than about 0.5% w/w of (S)—BINAP—RuCl₂ with respect to methyl 4-(2,4,5-trifluorophenyl)-3-oxobutanoate to obtain 3(S)-4-(2,4,5-trifluoro phenyl)-3-hydroxybutanoic acid; and
b) converting said 3(S)-4-(2,4,5-trifluorophenyl)-3-hydroxybutanoic acid to Sitagliptin or pharmaceutically acceptable salts thereof.

Preferably, hydrogenation in step a) is carried out at a temperature of about 60° C. to 80° C. and pressure of about 60 to 80 psi for 5 to 6 hours in presence of organic acid selected from acetic acid, formic acid, citric acid, lactic acid or tartaric acid.

According to another aspect of the present invention, conversion of 3(S)-4-(2,4,5-trifluorophenyl)-3-hydroxybutanoic acid to Sitagliptin or pharmaceutically acceptable salts thereof comprises the steps of,
a) treating said 3(S)-4-(2,4,5-trifluorophenyl)-3-hydroxybutanoic acid with protected hydroxylamine in presence of a coupling agent followed by cyclocondensation in presence of phosphine ligand and azodicarboxylate to obtain N-Benzyloxy-4(R)-[1-methyl-(2,4,5-trifluorophenyl)]-2-oxoazetidine;
b) subjecting said N-Benzyloxy-4(R)-[1-methyl-(2,4,5-trifluorophenyl)]-2-oxoazetidine to ring opening followed by treatment with 3-(trifluoromethyl)-5H,6H,7H,8H-[1,2,4]triazolo[4,3-a]pyrazine or its salt in presence of a coupling agent to obtain 3(R)-3-[(benzyloxy)amino]-1-[3-(trifluoromethyl)-5H, 6H,7H,8H-[1,2,4]triazolo[4,3-a]pyrazin-7-yl]-4-(2,4,5-trifluorophenyl) butan-1-one;
c) subjecting said 3(R)-3-[(benzyloxy)amino]-1[3-(trifluoromethyl)-5H,6H, 7H,8H-[1,2,4]triazolo[4,3-a]pyrazin-7-yl]-4-(2,4,5-trifluorophenyl)butan-1-one to debenzyloxylation in presence of a suitable catalyst and additive to obtain Sitagliptin; and
d) optionally converting said Sitagliptin into its pharmaceutically acceptable salts thereof, which is optionally purified.

Preferably, coupling agent is selected from N,N′-dicyclohexylcarbodiimide, 1-ethyl-3-(3-dimethyl amino propyl) carbodiimide or N,N′-diisopropylcarbodiimide; said phosphine ligand is selected from triphenylphosphine, tri(o-tolyl)phosphine, tributylphosphine or trioctylphosphine; and said azodicarboxylate is selected from diisopropylazodicarboxylate (DIAD), diethylazodicarboxylate (DEAD) or dibenzylazodicarboxylate.

Another aspect of the present invention provides process for preparation of Sitagliptin or pharmaceutically acceptable salts thereof comprising the steps of,
a) debenzyloxylation of 3(R)-3-[(benzyloxy)amino]-1-[3-(trifluoromethyl)-5H,6H,7H,8H-[1,2,4]triazolo [4,3-a]pyrazin-7-yl]-4-(2,4,5-trifluorophenyl) butan-1-one using hydrogen gas in presence of a catalyst and an additive to obtain a reaction mixture, wherein said additive is selected from benzyl chloride, benzyl bromide, benzyl iodide or substituted derivatives thereof;
b) isolating Sitagliptin from said reaction mixture.

Preferably, debenzyloxylation of 3(R)-3-[(benzyloxy)amino]-1-[3-(trifluoromethyl)-5H, 6H,7H,8H-[1,2,4]triazolo[4,3-a]pyrazin-7-yl]-4-(2,4,5-trifluorophenyl)butan-1-one is carried out in presence of palladium on carbon support and additive selected from benzyl chloride, benzyl bromide, benzyl iodide or substituted derivatives thereof; to obtain a reaction mixture. The obtained reaction mixture is further treated with trithiocyanuric acid to reduce the palladium content prior to isolation of Sitagliptin.

Another aspect of the present invention provides conversion of Sitagliptin to pharmaceutically acceptable salts thereof comprising the steps of,
a) dissolving Sitagliptin in a suitable solvent selected from methanol, ethanol, n-propanol, isopropanol, butanol, water, acetone, 2-butanone, diethyl ketone, diethyl ether, diisopropyl ether, ethyl acetate, methyl acetate, propyl acetate, butyl acetate or mixture thereof to obtain a solution;
b) adding salt forming agent selected from phosphoric acid, HCl, SOCl₂, NH₄Cl, HBr, methane sulfonic acid or ethane sulfonic acid to said solution to obtain a mixture;
c) optionally adding an antisolvent to said mixture; and
d) isolating pharmaceutically acceptable salts of Sitagliptin from said mixture.

Preferably, salt forming agent is phosphoric acid; said pharmaceutically acceptable salt is Sitagliptin phosphate; and said suitable solvent is selected from ethanol, isopropanol, water or mixture thereof.

Preferably, salt forming agent is HCl; said pharmaceutically acceptable salt is Sitagliptin HCl; and said suitable solvent is selected from acetone, 2-butanone, diethyl ketone, diethyl ether, isopropanol or mixture thereof.

Preferably, said salt forming agent is ethane sulfonic acid; said pharmaceutically acceptable salt is Sitagliptin esylate; and said suitable solvent is selected from methanol, ethanol, n-propanol, isopropanol, butanol, ethyl acetate, methyl acetate, propyl acetate, butyl acetate or mixture thereof; and said anti solvent is selected from diisopropyl ether, diethyl ether, methyl tert-butyl ether, THF or 1,4-dioxane.

Another aspect of the present invention provides Sitagliptin substantially free of impurity selected from group consisting of 3(R)-3-Amino-1-[3-(trifluoromethyl)-5H, 6H,7H,8H-[1,2,4]triazolo[4,3-a]pyrazin-7-yl]-4-(2,5-difluorophenyl)butan-1-one; 3(R)-3-Amino-1-[3-(trifluoromethyl)-5H,6H,7H,8H-[1,2,4]triazolo[4,3-a]pyrazin-7-yl]-4-(2,4-difluorophenyl)butan-1-one; and 3(R)-3-Amino-1-[3-(trifluoro methyl)-5H,6H,7H,8H-[1,2,4]triazolo[4,3-a]pyrazin-7-yl]-4-(3,4-difluorophenyl) butan-1-one.

Yet another aspect of the present invention provides isolated compound selected from the group consisting of 3(R)-3-Amino-1-[3-(trifluoromethyl)-5H,6H,7H,8H-[1,2,4]triazolo[4,3-a]pyrazin-7-yl]-4-(2,5-difluorophenyl)butan-1-one; 3(R)-3-Amino-1-[3-(trifluoromethyl)-5H,6H,7H,8H-[1,2,4]triazolo[4,3 -a]pyrazin-7-yl]-4-(2,4-difluorophenyl) butan-1-one; and 3(R)-3-Amino-1-[3-(trifluoromethyl)-5H, 6H,7H,8H-[1,2,4]triazolo[4,3-a]pyrazin-7-yl]-4-(3,4-difluorophenyl)butan-1-one.

Preferably, said isolated compound is used as a reference marker and/or reference standard in determining the purity of a sample of Sitagliptin or a pharmaceutical dosage form comprising Sitagliptin.

Another aspect of the present invention provides Sitagliptin HC1 characterized by X-ray diffraction pattern having peaks at 2-theta values of about 6.50, 7.96, 13.69, 16.01, 17.97, 18.61, 19.72, 20.26, 22.56, 24.63, 25.35, 25.60, 26.98, 29.31 and 31.54 degrees; or X-ray diffraction pattern having peaks at 2-theta values of about 5.18, 10.36, 12.72, 15.59, 16.06, 16.64, 17.27, 17.54, 19.85, 22.54, 23.62, 23.86, 24.22, 25.72, 26.25, 26.94, 28.09, 28.33, and 28.64 degrees; or X-ray diffraction pattern having peaks at 2-theta values of about 7.30, 7.89, 11.80, 15.77, 16.48, 17.86, 18.09, 20.30, 20.51, 20.88, 21.45, 24.05, 24.71, 25.16 and 25.50 degrees.

Another aspect of the present invention provides Sitagliptin esylate characterized by X-ray diffraction pattern having peaks at 2-theta values of about 6.83, 10.66, 12.10, 13.30, 13.67, 15.09, 15.65, 17.22, 18.41, 20.55, 21.49, 22.49, 24.36, 25.71, 27.34, 27.84 and 28.34 degrees; or X-ray diffraction pattern having peaks at 2-theta values of about 5.27, 10.63, 14.92, 15.51, 16.85, 19.26, 21.32, 22.48, 23.35, 24.17, 24.36, 25.23, 25.53 and 32.20 degrees; or X-ray diffraction pattern having peaks at 2-theta values of about 6.86, 13.73, 16.47, 20.60, 23.04, 26.89, 27.83, 33.82 and 34.66 degrees.

### DETAILED DESCRIPTION OF THE INVENTION

The present invention provides simple and industrially feasible process for preparation of Sitagliptin or pharmaceutically acceptable salts thereof. According to one embodiment of the present invention, there is provided a process for preparation of Sitagliptin or pharmaceutially acceptable salts thereof comprising the steps of,
a) hydrogenating methyl 4-(2,4,5-trifluorophenyl)-3-oxobutanoate in presence of less than about 0.5% w/w of (S)—BINAP—RuCl₂ with respect to methyl 4-(2,4,5-trifluorophenyl)-3-oxobutanoate to obtain 3(S)-4-(2,4,5-trifluoro phenyl)-3-hydroxybutanoic acid; and
b) converting said 3(S)-4-(2,4,5-trifluorophenyl)-3-hydroxybutanoic acid to Sitagliptin or pharmaceutically acceptable salts thereof.

Preferably hydrogenation in step a) is carried out at a temperature of about 60 to 80° C. and pressure of about 60 to 80 psi for about 5 to 6 hours in presence of a suitable organic acid selected from acetic acid, formic acid,citric acid, lactic acid or tartaric acid, preferably acetic acid and suitable solvent selected from methanol, ethanol, n-propanol, isopropanol, butanol, water or mixture thereof.

Another embodiment of the present invention provides conversion of 3(S)-4-(2,4,5-trifluorophenyl)-3-hydroxybutanoic acid to Sitagliptin or pharmaceutically acceptable salts thereof comprising the steps of,
a) treating 3(S)-4-(2,4,5-trifluorophenyl)-3-hydroxybutanoic acid with protected hydroxylamine in presence of a coupling agent followed by cyclocondensation in presence of phosphine ligand and azodicarboxylate to obtain N-Benzyloxy-4(R)-[1-methyl-(2,4,5-trifluorophenyl)]-2-oxoazetidine;
b) subjecting said N-Benzyloxy-4(R)-[1-methyl-(2,4,5-trifluorophenyl)]-2-oxoazetidine to ring opening followed by treatment with 3-(trifluoromethyl)-5H, 6H,7H,8H-[1,2,4]triazolo[4,3-a]pyrazine or its salt in presence of a coupling agent to obtain 3(R)-3-[(benzyloxy)amino]-1-[3-(trifluoromethyl)-5H, 6H,7H,8H-[1,2,4]triazolo[4,3-a]pyrazin-7-yl]-4-(2,4,5-trifluorophenyl) butan-1-one;
c) subjecting said 3(R)-3-[(benzyloxy)amino]-1-[3-(trifluoromethyl)-5H, 6H,7H,8H-[1,2,4]triazolo[4,3-a]pyrazin-7-yl]-4-(2,4,5-trifluorophenyl) butan-1-one to debenzyloxylation in presence of a suitable catalyst and additive to obtain Sitagliptin; and
d) optionally converting said Sitagliptin into its pharmaceutically acceptable salts thereof, which is optionally purified.

Said coupling agent is selected from N,N′-dicyclohexylcarbodiimide(DCC), 1-ethyl-3-(3-dimethylaminopropyl) carbodiimide (EDC), N,N′-diisopropylcarbodiimide(DIC); Said phosphine ligand is selected from triphenylphosphine, tri(o-tolyl)phosphine, tributylphosphine or trioctylphosphine; Said azodicarboxylate is selected from diisopropyl azodicarboxylate (DIAD), diethylazodicarboxylate (DEAD) or dibenzylazodicarboxylate; Said suitable catalyst is selected from palladium, platinum, rhodium or nickel on supports such as carbon, silica or alumina, oxides thereof or salts thereof; and Said additive is selected from benzyl chloride, benzyl bromide, benzyl iodide or substituted derivatives thereof.

Preferably, debenzyloxylation in step c) is carried out using hydrogen gas at a temperature of about 30 to 50° C. and pressure of about 30 to 50 psi for about 3 to 6 hours.

According to a preferred embodiment of the present invention, the process for preparation of Sitagliptin comprises the steps of,

Step I involves hydrogenation of methyl 4-(2,4,5-trifluorophenyl)-3-oxobutanoate (Formula III) in presence of less than 0.5% w/w of (S)—BINAP—RuCl₂ with respect to methyl 4-(2,4,5-trifluorophenyl)-3-oxobutanoate in presence of acetic acid and methanol in an autoclave to obtain a reaction mixture. The reaction mixture is subjected to hydrogenation at temperature of about 60° C. to 80° C., preferably at 70° C. and pressure of about 60 to 80 psi, preferably at 70 psi for about 5 to 6 hours. After the completion of reaction, the obtained solution is charged to a round bottom flask(RBF) followed by addition of water and aqueous sodium hydroxide at room temperature, preferably at 20° C. to 25° C. to obtain a mixture. The mixture is stirred for 60 to 120 min, preferably for 90 min. Methanol is removed from the mixture by distillation under vacuum followed by extraction of the resulting mixture with ether selected from methyl tert butyl ether (MTBE), diethyl ether, 1,4-dioxane, tetrahydrofuran (THF), dimethoxyethane (DME), diethoxyethane or mixture thereof, preferably methyl tert butyl ether. The layers are separated. The aqueous layer is cooled to 5° C. to 20° C., preferably to 10° C. to 15° C., acidified with conc. HCl and stirred for 1 to 3 hours, preferably for 2 hours to obtain slurry which is filtered to obtain a cake which is washed and dried to obtain 3(S)-4-(2,4,5-Trifluorophenyl)-3-hydroxybutanoic acid (Formula IV).

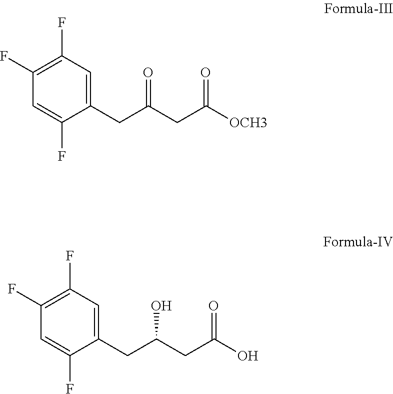 [SMILES: COC(=O)CC(=O)Cc1cc(F)c(F)cc1F ; O=C(O)C[C@@H](O)Cc1cc(F)c(F)cc1F]

Prior art discloses the use of about 0.8% w/w or more than 0.8% w/w of (S)—BINAP—RuCl₂ with respect to methyl 4-(2,4,5-trifluorophenyl)-3-oxobutanoate for the enantioselective hydrogenation of methyl 4-(2,4,5-trifluorophenyl)-3-oxo butanoate. Hydrogenation reaction is carried out at temperature of about 80° C. and at pressure of about 90 psi for about 10 hours. Besides, it has been reported in the prior art that 2N HCl is used along with (S)—BINAP—RuCl₂ during hydrogenation reaction to reduce the reaction time from 10 hours to 5 hours at high pressure, preferably 150 psi. It has been found by the inventors of the present invention that hydrogenation can be carried out at temperature of about 70° C. and pressure of about 70psi by using less than 0.5% w/w of (S)—BINAP—RuCl₂ with respect to methyl 4-(2,4,5-trifluorophenyl)-3-oxobutanoate thereby making the process cost effective.

The reaction tends to complete in about 5 to 6 hours thereby reducing the reaction time. Use of mineral acids such as HCl, as in the prior art, is not advisable since it can damage the reaction vessel.

Step II involves treatment of 3(S)-4-(2,4,5-Trifluorophenyl)-3-hydroxybutanoic acid with protected hydroxylamine in presence of a coupling agent in a suitable solvent selected from THF, 1,4-dioxane, diethyl ether or diisopropyl ether preferably THF followed by cyclocondensation in presence of phosphine ligand and azodicarboxylate to obtain N-(Benzyloxy)-4(R)-[1-methyl-(2,4,5-trifluorophenyl)]-2-oxoazetidine. 3(S)-4-(2,4,5-trifluorophenyl)-3-hydroxybutanoic acid, O-benzyl hydroxylamine hydrochloride, lithium hydroxide in THF and water are stirred at temperature of about 18° C. to 25° C., preferably at 20° C. to 22° C. to obtain a reaction mixture. DCC is added to the obtained reaction mixture in one lot and the suspension is stirred for 2 to 4 hours, preferably for 3 hours. After the completion of the reaction, the reaction mixture is diluted with solvent selected from MTBE, THF, 1,4-dioxane, diethyl ether or diisopropyl ether, preferably MTBE and filtered to obtain solid. The obtained solid is washed and the filtrate is subjected to layer separation. The organic layer is concentrated to obtain residue. The obtained residue is stripped with THF until all of the MTBE is removed and until KF of the solution is less than 0.2% as judged by Karl Fisher titration.The residue is diluted with THF and the final volume of the mixture is adjusted to the required volume.

This solution of hydroxamate is slowly added to a mixture of phosphine ligand, preferably triphenylphosphine in THF and diisopropylazodicarboxylate(DIAD) is added to the obtained mixture by maintaining the temperature below 10° C. over a period of 20 to 40 min, preferably 30 min to obtain a reaction mixture. After completion of addition, the reaction mixture is warmed to about 20° C. and stirred for 15 to 20 hours, preferably for 18 hours followed by addition of acetic acid. The mixture is concentrated under vacuum to obtain a residue. The obtained residue is cooled to 20 to 30° C., preferably 25° C. followed by addition of methanol and water. The obtained solution is cooled to −15° C. to −22° C., preferably −20° C. to obtain a slurry. The obtained slurry is stirred for 1 to 3 hours, preferably 2 hours at the same temperature and filtered to obtain a solid. The obtained solid is washed and dried to obtain compound of Formula V.

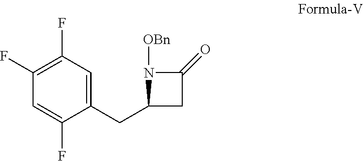 [SMILES: O=C1C[C@@H](Cc2cc(F)c(F)cc2F)N1OCc1ccccc1]

Prior art discloses the use of EDC-HCl as coupling agent. The present invention uses DCC as the coupling agent. DCC is much cheaper than EDC-HCl and hence use of DCC as coupling agent reduces the cost of the process by 1/10^{th} thereby making the process cost effective.

Step III involves ring opening of N-(Benzyloxy)-4(R)-[1-methyl-(2,4,5-trifluorophenyl)]-2-oxoazetidine in solvent selected from THF, 1,4-dioxane, acetonitrile, MTBE, water or mixture thereof, preferably THF and water using base selected from lithium hydroxide, potassium hydroxide, sodium hydroxide, sodium carbonate, potassium carbonate, sodium bicarbonate, potassium bicarbonate, sodium hydride, potassium hydride and the like, preferably lithium hydroxide at 15 to 30° C., preferably 20° C. to 25° C. over a period of 10 to 30 min, preferably 20 min to obtain a mixture. The obtained mixture is stirred at same temperature for 1 to 3 hours, preferably for 2 hours. The pH of the mixture is adjusted to about 3 using methanesulfonic acid maintaining the temperature below 20° C. The obtained mixture is subjected to extraction with MTBE or diethyl ether and layers are separated. The organic layer is concentrated to obtain a thick oil. The obtained oil is diluted with a solvent selected from acetonitrile, propionitrile, dimethylformamide (DMF), dimethylacetamide, N-methylacetamide, N-methylformamide, preferably acetonitrile followed by addition of triazole HCl .i.e., 1 3-trifluoromethyl[1,2,4 ]triazolo[4,3-a]piperazine HCl (VI) to obtain a mixture. The obtained mixture is cooled to −5° C. to 10° C., preferably 0° C. to 5° C. and N-methyl morpholine is added to the cold mixture followed by stirring at the same temperature. The obtained reaction mixture is charged with EDC-HCl and stirred at the same temperature for 1 to 4 hours, preferably for 3 hours. After the completion of reaction, the reaction mixture is diluted with water and MTBE. The layers are separated. The organic layer is washed and concentrated to obtain thick oil followed by dilution with alcohol, preferably ethanol. The alcoholic solution is taken up for further reaction.

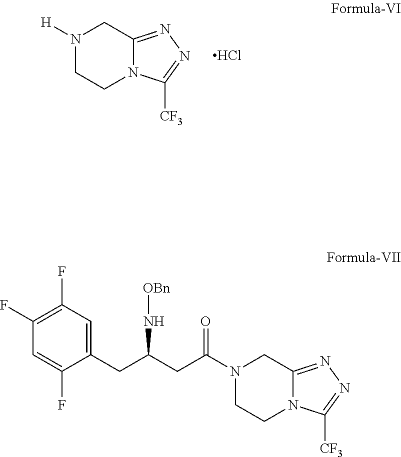 [SMILES: FC(F)(F)c1nnc2n1CCNC2 ; Cl ; O=C(C[C@@H](Cc1cc(F)c(F)cc1F)NOCc1ccccc1)N1CCn2c(nnc2C(F)(F)F)C1]

Step IV involves debenzyloxylation of 3(R)-3-[(benzyloxy)amino]-1-[3-(trifluoromethyl)-5H, 6H,7H,8H[1,2,4]triazolo[4,3-a]pyrazin-7-yl]-4-(2,4,5-trifluorophenyl) butan-1-one in presence of palladium catalyst on carbon support, Pd/C and additive selected from benzyl chloride, benzyl bromide, benzyl iodide, or substituted derivatives thereof, preferably benzyl chloride to obtain Sitaglipin phosphate.

An ethanolic solution of 3(R)-3-[(benzyloxy)amino]-1-[3-(trifluoromethyl)-5H,6H, 7H,8H[1,2,4]triazolo[4,3-a]pyrazin-7-yl]-4-(2,4,5-trifluorophenyl)butan-1-one, ethanol and water are subjected to debenzyloxylation in presence of Pd/C, preferably 10% Pd/C and benzyl chloride, preferably 0.1 to 3 equivalents of benzyl chloride with respect to 3(R)-3-[(benzyloxy)amino]-1-[3-(trifluoromethyl)-5H,6H, 7H,8H[1,2,4]triazolo[4,3-a]pyrazin-7-yl]-4-(2,4,5-trifluorophenyl)butan-1-one in an autoclave with pressure of about 30 to 50 psi, preferably 40 psi and the mixture is stirred at temperature of about 30° C. to 50° C., preferably at 40° C. for 3 to 6 hours, preferably for 4 to 5 hours. The obtained mixture is filtered and the filtrate is concentrated to get an oil. The obtained oil is diluted with water and to this is added a scavenger selected from trithiocyanuric acid, EDTA, alumina, silica gel, polymer supported thiourea or aliphatic thio compound, preferably trithiocyanuric acid. The mixture is stirred for 1 to 3 hours, preferably 2 hours at 25° C. to 35° C. and filtered to obtain a solid. The obtained solid is washed with water and pH of the filtrate is adjusted to 13. This reaction mixture is then subjected to extraction using a mixture of solvents, such as MTBE and acetonitrile. The layers are separated and aqueous layer is extracted with MTBE . The combined organic layers are concentrated to obtain Sitagliptin base as oil.

Step V involves conversion of obtained Sitagliptin base to desired pharmaceutically acceptable salt using pharmaceutically acceptable acid with/without isolating Sitagliptin base. The process of the present invention is preferably carried out without isolating Sitagliptin base.

Preferably, obtained Sitagliptin base, solvent selected from methanol, ethanol, n-propanol, isopropanol, butanol, water or mixture thereof are charged in a RBF to obtain a mixture. To the obtained mixture is added aqueous phosphoric acid and the mixture is heated to temperature of about 65° C. to 85° C., preferably 75° C. to obtain a clear solution. The obtained clear solution is cooled to 60° C. to 70° C., preferably 65° C. to 68° C. and stirred for 1 to 3 hours, preferably for 2 hours. The solution is further cooled to 50° C. to 65° C., preferably 55° C. to 60° C. followed by seeding with Sitagliptin phosphate to obtain a slurry. The obtained slurry is cooled at room temperature, preferably 25° C. followed by addition of solvent selected from isopropanol, methanol, ethanol, n-propanol or butanol and stirred for 10 to 15 hours, preferably 12 hours. The slurry is filtered, washed and dried to obtain Sitagliptin phosphate monohydrate as solid.

Step VI involves the purification of Sitagliptin phosphate monohydrate by treating crude Sitagliptin phosphate monohydrate with solvent selected from isopropanol(IPA), methanol, ethanol, n-propanol, butanol, water or mixture thereof to obtain a slurry. The slurry is heated to a temperature of about 70° C. to 80° C. , preferably 75° C. to obtain a solution. The solution is cooled to 25° C. to 30° C. and seeded with Sitagliptin phosphate monohydrate at temperature of about 50° C. to 65° C., preferably 55° C. to 60° C. followed by addition of alcohol, preferably IPA over a period of 1 hour. The mixture is stirred for 12 hours and filtered to obtain a solid which is washed to obtain pure Sitagliptin phosphate monohydrate.

Prior art discloses the use of additives such as HCl, HBr, acetic acid and formic acid during the debenzyloxylation of compound (VII) under hydrogenolytic conditions in presence of metal catalyst, preferably palladium in methanol as solvent. The inventors of the present invention have surprisingly found that using additives such as benzyl halide controls the defluorination reaction thereby minimizing the desfluoro impurities well below 0.15% thereby producing pure Sitagliptin and salts thereof. Use of mineral acids such as HCl are not advisable considering equipment safety.

Besides, the use of commercially available and cheap trithiocyanuric acid in small amounts enables easy removal of palladium impurities. It has been found that palladium content level gets reduced from ˜500 ppm to ˜1 ppm making the process industrially viable.

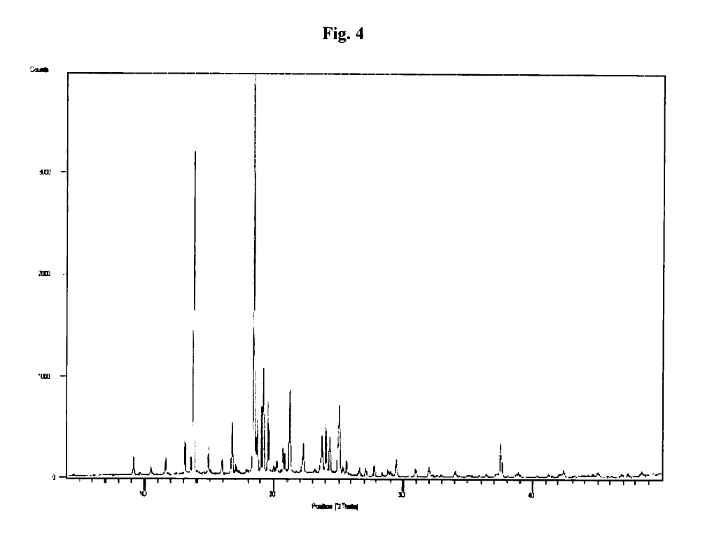

The obtained Sitagliptin phosphate monohydrate is characterized by X-ray diffrac-tion pattern as shown in FIG. 4 Sitagliptin phosphate monohydrate is further characterized by X-ray diffraction pattern having peaks expressed as 2-theta values of about 9.15, 10.48, 11.68, 13.18, 13.78, 14.89, 15.96, 16.75, 17.07, 18.43 18.71, 19.12, 19.54, 20.23, 20.76, 21.13, 22.27, 23.69, 24.01, 24.35, 25.02, 25.65, 26.62, 27.14, 27.84, 28.32, 28.79, 29.47, 30.96, 31.98, 34.04, 37.45, 38.81, 41.21, 42.35, 45.02 and 48.43 degrees.

The process for preparation of Sitagliptin phosphate according to the present invention is illustrated by the following reaction scheme:

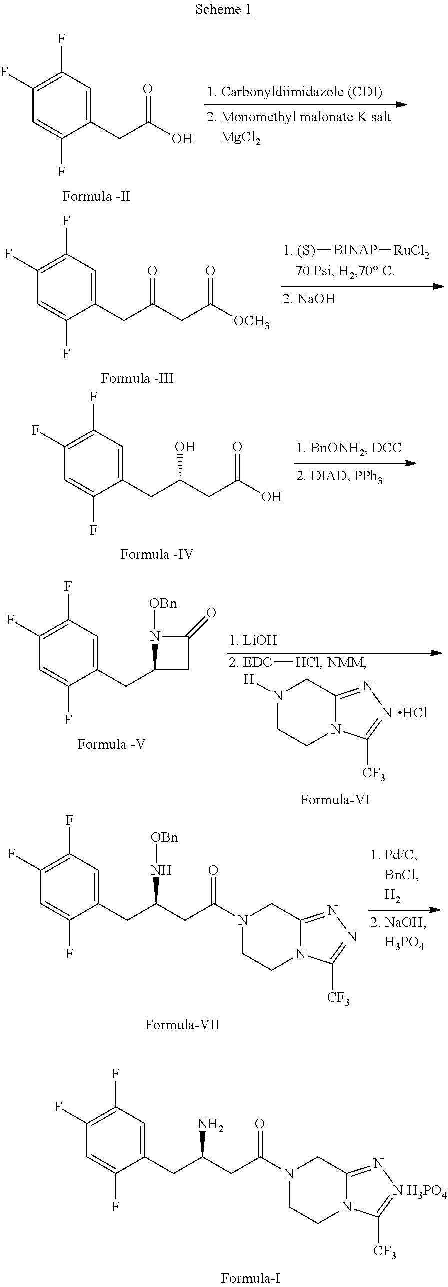

Another embodiment of the present invention provides process for preparation of Sitagliptin or pharmaceutically acceptable salts thereof comprising the steps of,
a) debenzyloxylation of 3(R)-3-[(benzyloxy)amino]-1-[3-(trifluoromethyl)-5H, 6H,7H,8H-[1,2,4]triazolo[4,3-a]pyrazin-7-yl]-4-(2,4,5-trifluoro phenyl) butan-1-one using hydrogen gas in presence of a catalyst and an additive to obtain a reaction mixture, wherein said additive is selected from benzyl chloride, benzyl bromide, benzyl iodide or substituted derivatives thereof;
b) isolating Sitagliptin from said reaction mixture.

Preferably, debenzyloxylation is carried out at a temperature of about 40° C. and pressure of about 40 psi for about 4 to 5 hours; and said catalyst is selected from palladium, platinum, rhodium or nickel on supports such as carbon, silica or alumina, oxides thereof or salts thereof. Preferably, the reaction mixture is further treated with trithiocyanuric acid to reduce the palladium content prior to isolation of Sitagliptin.

Methyl 4-(2,4,5-trifluorophenyl)-3-oxobutanoate (Formula III) used in the synthesis of Sitagliptin can be synthesized by any process known in the art.

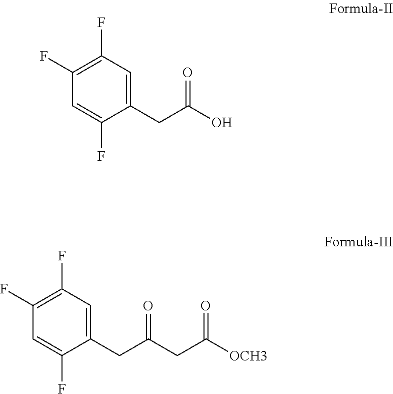 [SMILES: O=C(O)Cc1cc(F)c(F)cc1F ; COC(=O)CC(=O)Cc1cc(F)c(F)cc1F]

Preferably, monomethylmalonate potassium salt, triethylamine and acetonitrile are charged to a 3 L round bottom flask (RBF) fitted with condenser, nitrogen inlet, thermometer pocket and overhead stirrer. MgCl₂ is added lot-wise to the above mixture over a period of 15-20 min at 30° C. and the mixture is stirred for 10 min. The reaction mixture is heated to 50° C. for 8 h at same temperature. After 8 hours, the reaction mixture is cooled to 30° C. and marked as Part A. 1,1′-carbonyldiimidazole (CDI) and acetonitrile are charged to a 2 L RBF fitted with nitrogen inlet, thermometer pocket, addition funnel and overhead stirrer. To this, a solution of 2,4,5-trifluorophenyl acetic acid (Formula II) in acetonitrile is added slowly over a period of a 30 min at 30° C. and the reaction mixture is stirred at 30° C. for 3 h. This solution is marked as part B.

The Part B solution is added drop-wise to part-A slurry over a period of 2 hour at 30° C. and the mixture is stirred. The progress of the reaction is monitored by TLC (Mobile phase: n-Hexane: ethyl acetate; 50:50). After 12 h, TLC analysis indicated <5% of un-reacted starting material. The reaction mixture is concentrated under reduced pressure at 50-55° C. to get a thick slurry. To this, water is added and the mixture is cooled to 10-15° C. and conc. HCl is added slowly, below 20° C. MTBE is added to the mixture and the mixture is stirred for 30 min. The layers are separated and the aqueous layer is extracted with MTBE (200 ml). The combined organic layers are washed with 7% aqueous NaHCO₃ solution (400 ml) followed by washing with brine. The organic layer is dried over sodium sulphate and concentrated under vacuum at 50° C. to get an oil. The obtained oil is diluted with isopropyl alcohol and cooled to 20° C. To this water is added slowly over a period of 4 hours at 18-20° C. The slurry is then stirred for 4 hours and filtered. The obtained cake is washed with water (100 ml) and then suck dried to obtain Methyl 4-(2,4,5-trifluorophenyl)-3-oxobutanoate.

In the process of the present invention, the reaction time for the synthesis of Methyl 4-(2,4,5-trifluorophenyl)-3-oxobutanoate is considerably reduced. Besides the work-up procedure is simplified as compared to the prior art.

The present inventors have invented a simple process for preparation of Sitagliptin minimizing the impurities formed during the process as compared to the prior art process, thereby making the process cost effective and commercially viable.

An alternate embodiment of the present invention provides process for preparation of Sitagliptin or salts thereof by a process represented in below scheme,

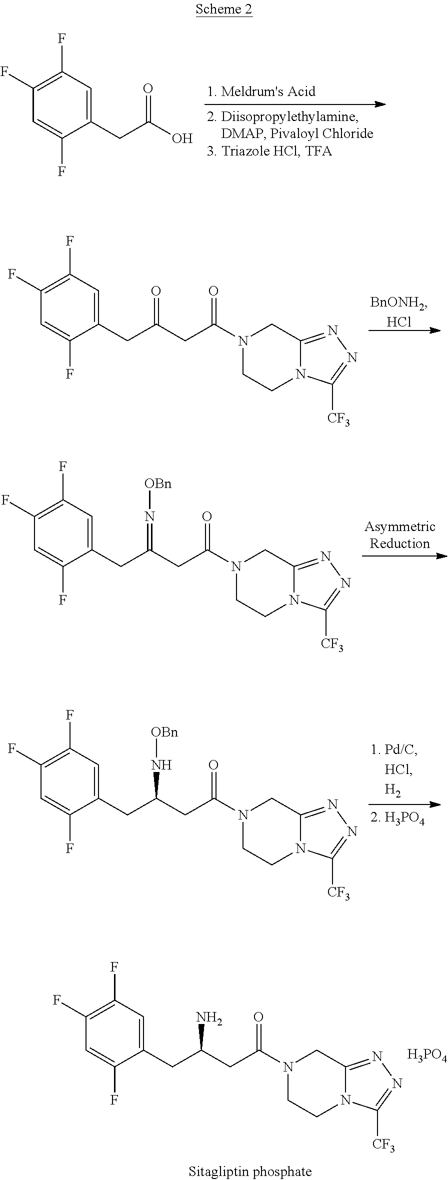

Another embodiment of the present invention provides Sitagliptin or pharmaceutically acceptable salts thereof substantially free of impurity selected from impurity A, namely 3(R)-3-Amino-1- [3-(trifluoromethyl)-5H,6H,7H,8H[1,2,4]triazolo[4,3-a]pyrazin-7-yl]-4-(2,5-difluorophenyl)butan-1-one; impurity B namely 3(R)-3-Amino-1-[3-(trifluoromethyl)-5H,6H,7H,8H[1,2,4]triazolo[4,3-a]pyrazin-7-yl]-4-(2,4-difluoro phenyl)butan-1-one and impurity C namely 3(R)-3-Amino-1-[3-(trifluoromethyl)-5H, 6H,7H,8H[1,2,4]triazolo[4,3-a]pyrazin-7-yl]-4-(3,4-difluorophenyl) butan-1-one.

Preferably, Sitagliptin or Sitagliptin phosphate obtained according to the present invention is substantially free of below mentioned impurities:

| Impurity | Structure | Name | Mass | RRT's |
|---|---|---|---|---|
| Impurity A | 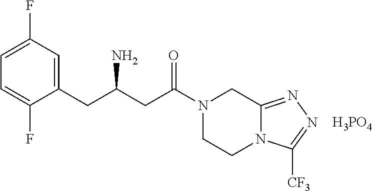 | (3R)-3-Amino-1-[3- (trifluoromethyl)- 5H,6H,7H,8H [1,2,4] triazolo [4,3- a]pyrazin-7-yl]-4- (2,5-difluorophenyl) butan-1-one phosphate | 389 | 0.75 ± 0.02 |
| Impurity B | 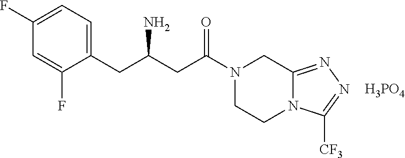 | (3R)-3-Amino-1-[3- (trifluoromethyl)- 5H,6H,7H,8H [1,2,4] triazolo [4,3- a]pyrazin-7-yl]-4- (2,4- difluorophenyl)butan- 1-one phosphate | 389 | 0.82 ± 0.02 |
| Impurity C | 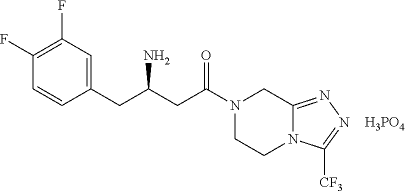 | (3R)-3-Amino-1-[3- (trifluoromethyl)- 5H,6H,7H,8H [1,2,4] triazolo [4,3- a]pyrazin-7-yl]-4- (3,4-difluorophenyl) butan-1-one phosphate | 389 | 0.89 ± 0.02 |

Yet another embodiment of the present invention provides isolated compound selected from the group consisting of 3(R)-3-Amino-1-[3-(trifluoromethyl)-5H, 6H,7H,8H-[1,2,4]triazolo[4,3-a]pyrazin-7-yl]-4-(2,5-difluorophenyl)butan-1-one; 3(R)-3 -Amino-1-[3-(trifluoromethyl)-5H,6H,7H,8H-[1,2,4]triazolo[4,3-a]pyrazin-7-yl]-4-(2,4-difluorophenyl)butan-1-one; and 3(R)-3 -Amino-1-[3-(trifluoromethyl)-5H, 6H,7H,8H-[1,2,4triazolo[4,3-a]pyrazin-7-yl]-4-(3,4-difluorophenyl) butan-1-one.

Another embodiment of the present invention provides the use of these isolated compound as reference marker and/or reference standard for determining the purity of Sitagliptin or pharmaceutical dosage form comprising Sitagliptin.

The conditions of HPLC method for isolating these impurities are given below;

Column: Zorbax XDB C8, 150 mm×4.6 mm, 5 μ

Eluent: Solvent A: 0.01M K2HPO₄ in H₂O pH 6.5 by orthophosphoric acid

Solvent B: Methanol

Isocratic: 50% A: 50% B

Flow rate: 1 min/ml

Injection Vol.: 10.0 uL,

UV detection: 210 nm

The general method for synthesizing these impurities is represented in below scheme,

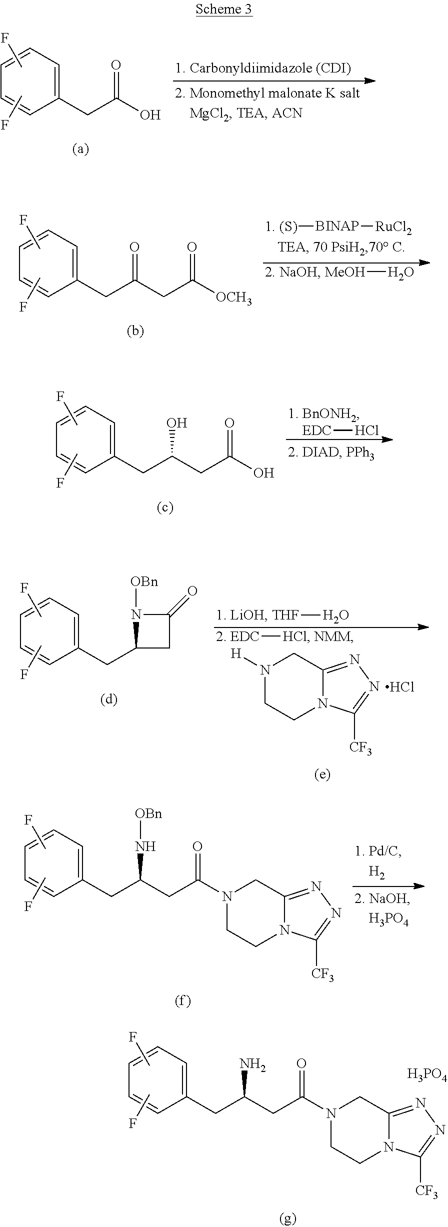

The compound (b) is prepared by combining corresponding difluorophenylacetic acid (a), with monomethyl malonate potassium salt in presence of carboxylic acid-activating reagent such as 1,1′-carbonyldiimidazole (CDI) in a suitable organic solvent. The compound (c) is prepared by combining compound (b) with an enantioselective catalyst in presence of hydrogen followed by treating with an aqueous base. The compound (d) is produced by combining compound (c) with benzylhy-droxylamine in the presence of a coupling reagent followed by cyclocondensing with an azodicarboxylate in the presence of a phosphine ligand. The compound (d) is converted to compound (f) by treating with an aqueous base followed by treatment with compound (e) (triazole hydrochloride compound), in presence of a coupling reagent. The compound (f) is subjected to debenzoxylation in presence of palladium catalyst to afford compound (g).

Another embodiment of the present invention provides an analytical method for testing the impurity profile of Sitagliptin phosphate. These methods are also suitable for analyzing different salts of Sitagliptin.

Quantitative analysis of Sitagliptin or salts thereof can be carried out using conventional analytical techniques, preferably HPLC.

In a preferred embodiment, the present invention provides an analytical method for detecting impurity A, impurity B and impurity C in a sample of Sitagliptin or pharmaceutically acceptable salts thereof which comprises the steps of
1. providing a sample of Sitagliptin or pharmaceutically acceptable salt thereof containing or suspected of containing impurity A, impurity B, impurity C or mixture thereof;
2. subjecting the sample of step a) to chromatographic technique, preferably HPLC and
3. determining the peak corresponding to impurity A, impurity B and impurity C.

Impurity A, impurity B and impurity C appear at RRT (relative retention time) of about 0.75±0.02, 0.82±0.02 and 0.89±0.02 respectively relative to Sitagliptin.

Sitagliptin phosphate obtained according to the present invention has purity more than 99.5% preferably 99.9% (by HPLC) and overall yield of about 36%.

Apart from potential process related impurities, there are some genotoxic impurities. The control of these impurities is more difficult while scaling-up the processes according to the prior art resulting in extensive isolation/purification procedures in order to obtain pure Sitagliptin or salts thereof. The process of the present invention provides Sitagliptin or salts thereof wherein these impurities are controlled well below 100 ppm thus complying the ICH guidelines.

Various genotoxic impurities which may be present in the final API are as described below,
a) 1-(Chloromethyl)-2,4,5-trifluorobenzene:

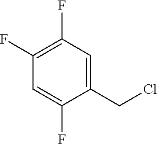 [SMILES: Fc1cc(F)c(CCl)cc1F]

1-(Chloromethyl)-2,4,5-trifluorobenzene impurity is generated during the synthesis of 2,4,5-trifluorophenylacetic acid, a starting material for the preparation of Sitagliptin.
b) N′-(2-Chloroacetyl)-2,2,2-trifluoroacetohydrazide:

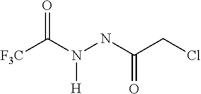 [SMILES: O=C(CCl)NNC(=O)C(F)(F)F]

c) 5-(trifluoromethyl)-2-(chloromethyl)- 1,3,4-oxadiazole:

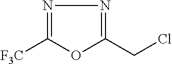 [SMILES: FC(F)(F)c1nnc(CCl)o1]

d) N-[(2Z)-piperazin-2-ylidene] trifluoroaceto hydrazide:

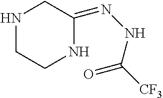 [SMILES: O=C(N/N=C1/CNCCN1)C(F)(F)F]

The above three impurities namely N′-(2-Chloroacetyl)-2,2,2-trifluoroaceto-hydrazide, 5-(trifluoromethyl)-2-(chloromethyl)-1,3,4-oxadiazole and N-[(2Z)-piperazin-2-ylidene]trifluoroaceto hydrazide are generated during synthesis of 3-trifluoromethyl[1,2,4]triazolo[4,3-a ]piperazine HCl, compound (VI).

Another embodiment of the present invention provides process for preparation of Sitagliptin which comprises,
a) suspending Sitagliptin salt in a suitable solvent to obtain a solution;
b) adding sodium hydroxide to the obtained solution; and
c) isolating Sitagliptin base.

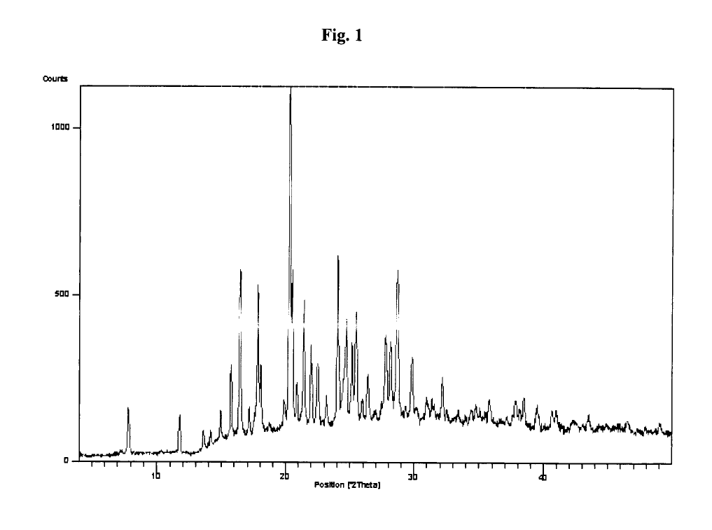

Preferably, Sitagliptin phosphate is suspended in suitable solvent preferably water. The obtained suspension is cooled to 0-5° C. and treated with sodium hydroxide fol-lowed by stirring for several hours to obtain Sitagliptin which is characterized by X-ray diffraction pattern as shown in FIG. 1. It is further characterized by X-ray diffraction pattern having peaks expressed as 2-theta values of about 7.82, 11.75, 13.60, 14.93, 15.67, 16.43, 17.15, 17.78, 18.05, 19.87, 20.27, 20.49, 20.87, 21.39, 21.96, 22.46, 23.17, 24.02, 24.70, 25.14, 25.44, 26.38, 27.74, 28.11, 28.66, 29.81, 32.17 and 38.47 degrees.

Another embodiment of the present invention provides process for preparation of Sitagliptin salts comprising the steps of,
a) dissolving Sitagliptin in a suitable solvent selected from methanol, ethanol, n-propanol, isopropanol, butanol, water, acetone, 2-butanone, diethyl ketone, diethyl ether, diisopropyl ether, ethyl acetate, methyl acetate, propyl acetate, butyl acetate or mixture thereof to obtain a solution;
b) adding salt forming agent selected from phosphoric acid, HCl, SOCl₂, NH₄Cl, HBr, methane sulfonic acid or ethane sulfonic acid to said solution to obtain a mixture;
c) optionally adding an antisolvent to said mixture; and
d) isolating pharmaceutically acceptable salts of Sitagliptin from said mixture.

Preferably, Sitagliptin base is treated with a suitable solvent selected from isopropanol, methanol, ethanol, n-propanol or butanol, preferably isopropanol to obtain a solution. The obtained solution is treated with concentrated hydrochloric acid at temperature of about 25° C. to 35° C., preferably 30° C. and stirred for 2 to 4 hours, preferably 3 hours to obtain a solid. The obtained solid is filtered, dried at temperature of about 50° C. to 70° C., preferably 60° C. to obtain Form III of Sitagliptin HCl.

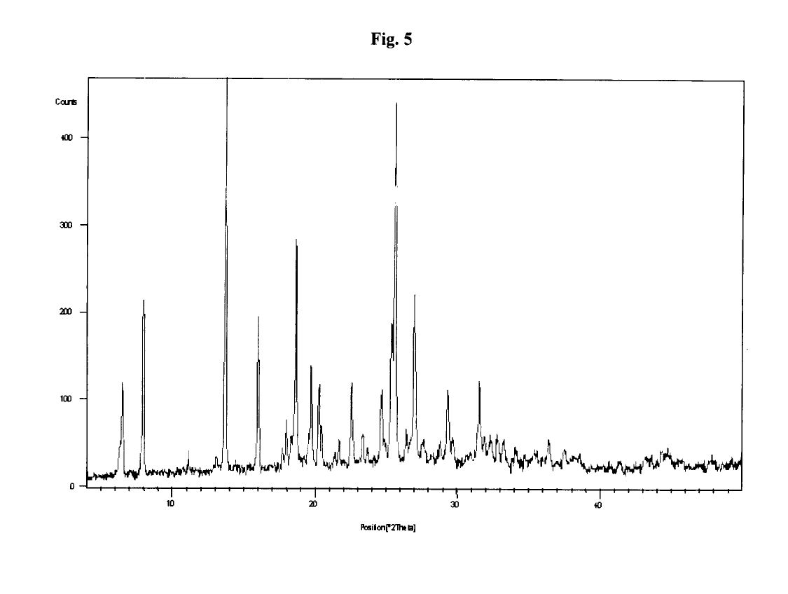

Another embodiment of the present invention provides Sitagliptin hydrochloride Form III characterized by X-ray diffraction pattern as shown in FIG. 5. It is further characterized by X-ray diffraction pattern having peaks expressed as 2-theta values of about 6.50, 7.96, 13.69, 16.01, 17.97, 18.61, 19.72, 20.26, 22.56, 24.63, 25.35, 25.60, 26.98, 29.31 and 31.54 degrees.

In a preferred embodiment, Sitagliptin HCl Form III is taken in a suitable solvent selected from acetone, 2-butanone or diethylketone preferably acetone and the mixture is heated at reflux to obtain a clear solution. The obtained hot solution is filtered and poured into antisolvent selected from diisopropyl ether, diethyl ether, methyl tert-butyl ether, THF or 1,4-dioxane, preferably diisopropyl ether at temperature of about 25° C. to 35° C., preferably 30° C. The obtained sticky mass is stirred at the same temperature for 4 to 6 hours, preferably 5 hours. The obtained solid is filtered and dried at temperature of about 50° C. to 60° C., preferably at 55° C. to obtain Form IV of Sitagliptin HCl.

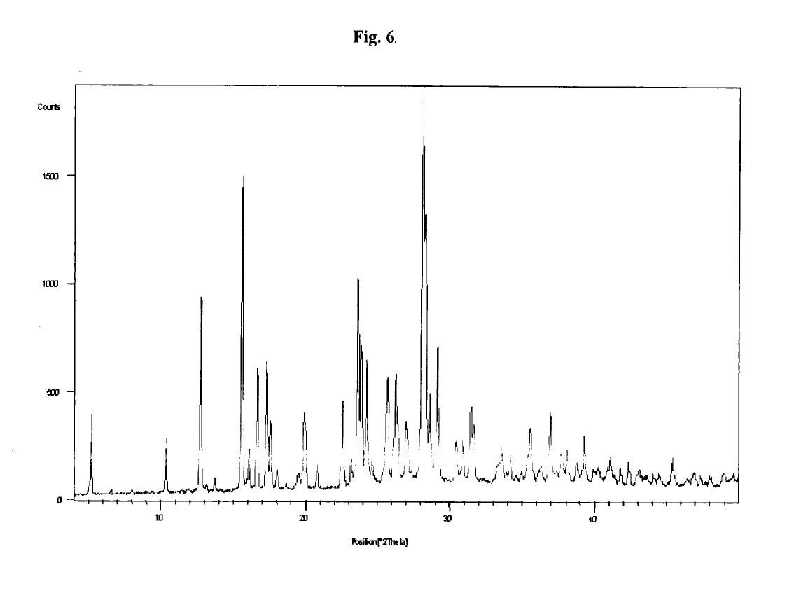

Another embodiment of the present invention provides Sitagliptin hydrochloride Form IV characterized by X-ray diffraction pattern as shown in FIG. 6. It is characterized by X-ray diffraction pattern having peaks expressed as 2-theta values of about 5.18, 10.36, 12.72, 15.59, 16.06, 16.64, 17.27, 17.54, 19.85, 22.54, 23.62, 23.86, 24.22, 25.72, 26.25, 26.94, 28.09, 28.33 and 28.64 degrees. It is further characterized by X-ray diffraction pattern having peaks expressed as 2-theta values of about 13.72, 18.01, 20.79, 23.15, 29.16, 30.44, 30.92, 31.46, 31.68, 33.60, 34.18, 35.56, 36.28, 36.94, 37.67, 38.06, 38.78, 39.30, 40.13,41.11, 41.80, 42.35, 43.03 and 45.40 degrees.

In a preferred embodiment, Sitagliptin base is suspended in a suitable solvent selected from diisopropyl ether, diethyl ether, methyl tert-butyl ether, THF or 1,4-dioxane, preferably diisopropyl ether at temperature of about 20° C. to 30° C., preferably 25° C. to obtain a solution. Dry HCl gas is passed through the obtained solution to attain pH in the range of 3 to 4 and stirred for 20 to 40 min, preferably 30 min to obtain Form V of Sitagliptin HCl.

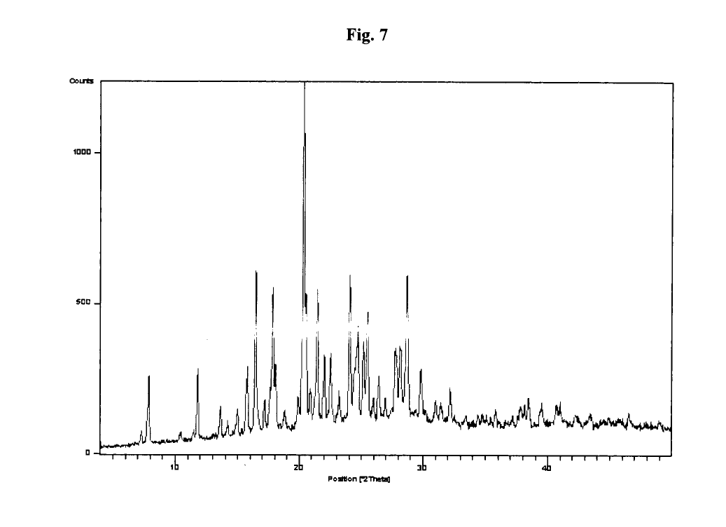

Another embodiment of the present invention provides Sitagliptin hydrochloride Form V characterized by X-ray diffraction pattern as shown in FIG. 7. It is characterized by X-ray diffraction pattern having peaks expressed as 2-theta values of about 7.30, 7.89, 11.80, 15.77, 16.48, 17.86, 18.09, 20.30, 20.51, 20.88, 21.45, 24.05, 24.71, 25.16 and 25.50 degrees. It is further characterized by X-ray diffraction pattern having peaks expressed as 2-theta of about 13.62, 14.98, 17.16, 18.77, 19.88, 21.99, 22.51, 23.19, 26.40, 27.74, 28.19, 28.68, 29.83, 31.00, 32.17, 38.45, 39.48 and 42.36 degrees.

In a preferred embodiment, ethane sulfonic acid is dissolved in a suitable solvent selected from isopropanol, methanol, ethanol, n-propanol or butanol, preferably isopropanol to obtain a solution. Sitagliptin base is added to the obtained solution and stirred at temperature of about 25° C. to 35° C., preferably 30° C. for 1 to 5 hours, preferably 3 hours to obtain Sitagliptin esylate Form I.

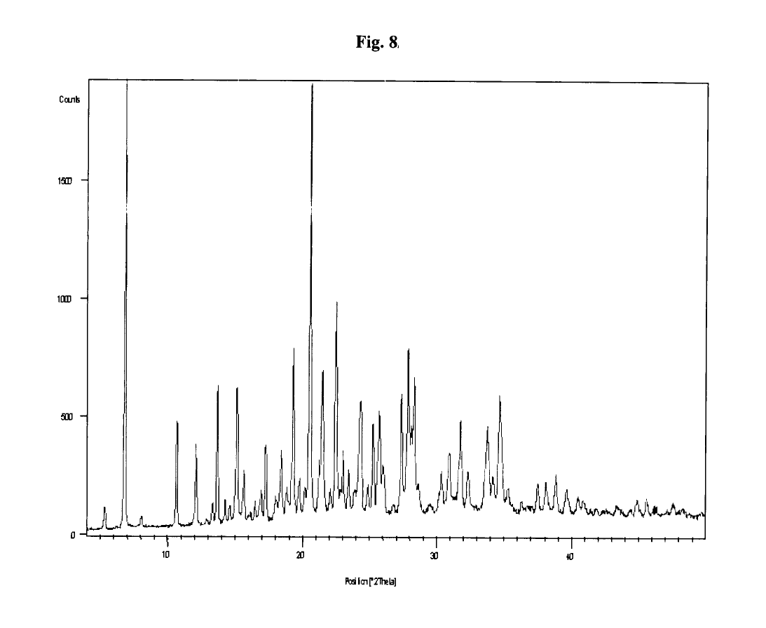

Another embodiment of the present invention provides Sitagliptin esylate Form I characterized by X-ray diffraction pattern as shown in FIG. 8. It is further characterized by X-ray diffraction pattern having peaks expressed as 2-theta values of about 6.83, 10.66, 12.10, 13.30, 13.67, 15.09, 15.65, 17.22, 18.41, 20.55, 21.49, 22.49, 24.36, 25.71, 27.34, 27.84 and 28.34 degrees. It is further characterized by X-ray diffraction pattern having peaks expressed as 2-theta values of about 5.33, 8.02, 14.24, 14.56, 16.46, 16.90, 18.79, 19.30 19.76, 21.20, 22.08, 23.00, 23.44, 24.88, 25.23, 26.02, 30.37, 30.96, 31.77, 32.34, 33.78, 34.16, 34.63 37.48, 67, 38.07, 38.84, 39.62, 40.52, 43.46, 44.86 and 45.60 degrees.

In a preferred embodiment, Sitagliptin esylate Form I is dissolved in a suitable solvent selected from methanol, ethanol, n-propanol, isopropanol, or butanol preferably methanol at temperature of about 50° C. to 70° C., preferably 60° C. to obtain clear solution. The obtained hot solution is filtered. Anti-solvent selected from diisopropyl ether, diethyl ether, methyl tert-butyl ether, THF or 1,4-dioxane, preferably diisopropyl ether is added to the filtrate at 25 to 30° C. to obtain a solution. The obtained solution is stirred for several hours at the same temperature to obtain Sitagliptin esylate Form II.

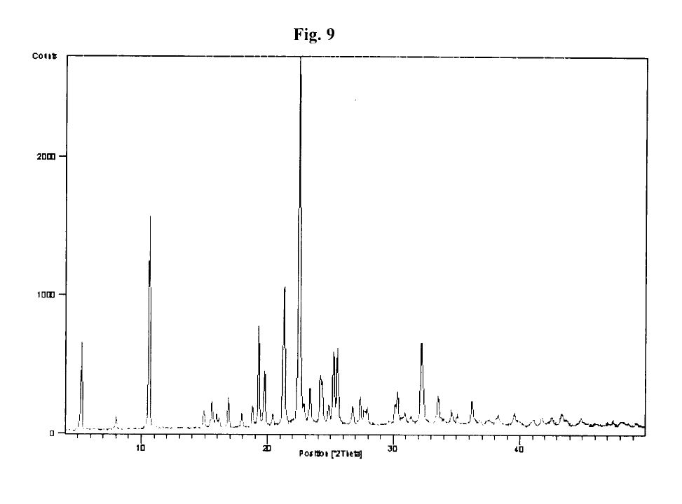

Another embodiment of the present invention provides Sitagliptin esylate Form II characterized by X-ray diffraction pattern as shown in FIG. 9. It is characterized by X-ray diffraction pattern having peaks expressed as 2-theta values of about 5.27, 10.63, 14.92, 15.51, 16.85, 19.26, 21.32, 22.48, 23.35, 24.17, 24.36, 25.23, 25.53 and 32.20 degrees. It is further characterized by X-ray diffraction pattern having peaks expressed as 2-theta values of about 8.01, 17.93, 18.78, 19.76, 20.42, 24.84, 25.53, 26.76, 27.38, 27.94, 30.13, 30.36, 30.92, 33.56, 34.60,35.04, 36.21, 38.27, 39.58, 41.78, 42.57 and 43.32 degrees.

In a preferred embodiment, Sitagliptin esylate Form I or Form II is suspended in a suitable solvent selected from ethyl acetate, methyl acetate, butyl acetate, propyl acetate, preferably, ethyl acetate and heated to reflux to obtain a clear solution. A second solvent selected from ethanol, methanol, n-propanol, isopropanol, or butanol preferably ethanol is added to the obtained clear solution. The solution is filtered and the filtrate is cooled to 25° C. and stirred for 2 to 5 hours, preferably for 3 hours to obtain Sitagliptin esylate Form III.

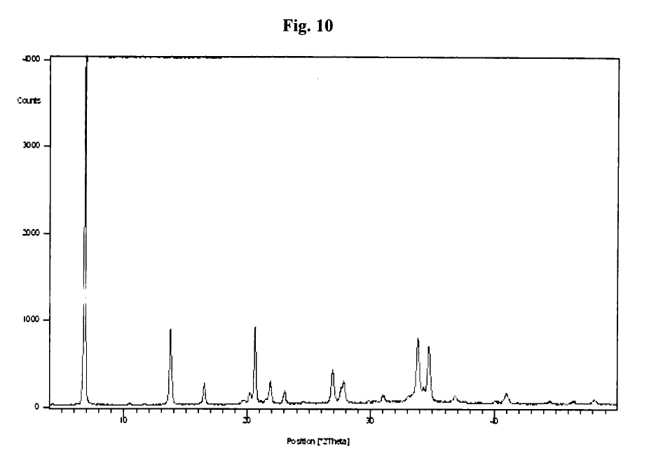

Another embodiment of the present invention provides Sitagliptin esylate Form III characterized by X-ray diffraction pattern as shown in FIG. 10. It is characterized by X-ray diffraction pattern having peaks expressed as 2-theta values of about 6.86, 13.73, 16.47, 20.60, 23.04, 26.89, 27.83, 33.82 and 34.66 degrees. It is further characterized by X-ray diffraction pattern having peaks expressed as 2-theta values of about 10.43, 20.18, 21.87, 31.03, 36.81, 40.88, 46.34 and 48.09 degrees.

Another embodiment of the present invention provides pharmaceutical composition comprising Sitagliptin or pharmaceutically acceptable salts thereof or any polymorph thereof and at least one pharmaceutically acceptable excipient. The pharmaceutical compositions may be prepared by any conventional techniques known in the art.

Another embodiment of the present invention provides Sitagliptin or pharmaceutically acceptable salts thereof having particle size distribution such that 90% particles have particle size less than about 100 microns, preferably less than about 50 microns. Preferably Sitagliptin phosphate obtained according to the present invention has particle size distribution such that 90% particles have particle size less than about 50 microns, preferably less than about 30 microns.

Unless otherwise indicated, the following definitions are set forth to illustrate and define the meaning and scope of the various terms used to describe the invention herein.

The term “substantially free” means Sitagliptin or pharmaceutically acceptable salts thereof having less than about 1%, preferably less than about 0.5%, more preferably less than about 0.3%, most preferably less than about 0.15% of impurities or other polymorphic forms.

As used herein, the term “isolated” refers to a compound that is at least 80%, preferably at least 90%, more preferably at least 95%, and most preferably at least 99% pure, as judged by GC or HPLC.

A “reference marker” is used in qualitative analysis to identify components of a mixture based upon their position, e.g. in a chromatogram or on a Thin Layer Chromatography (TLC) plate (Strobel pp. 921, 922, 953). For this purpose, the compound does not necessarily have to be added to the mixture if it is present in the mixture. A “reference marker” is used only for qualitative analysis, while a reference standard may be used for quantitative or qualitative analysis, or both.

As used herein, the term “reference standard” refers to a compound that may be used for both, quantitative and qualitative analysis of an active pharmaceutical ingredient.

The following examples illustrate the invention described above; however, they are not intended to limit its extent in any manner.

### Example 1

### Step-1

### Preparation of Methyl 4-(2,4,5-trifluorophenyl)-3-oxobutanoate (Formula III)

Monomethylmalonate potassium salt (MMMKS; 122.8 g), triethylamine (264 ml) and acetonitrile (1200 ml) were charged to a 3 L round bottom flask (RBF) fitted with condenser, nitrogen inlet, thermometer pocket and overhead stirrer. MgCl₂ (65.2 g) was added lot-wise to the above mixture over a period of 15-20 min at 30° C. and the mixture was stirred for 10 min. The reaction mixture was heated to 50° C. for 8 h at same temperature. After 8 hours, the reaction mixture was cooled to 30° C. and marked as Part A. 1,1′-carbonyldiimidazole (CDI) (110 g) and acetonitrile (250 ml) were charged to a 2 L RBF fitted with nitrogen inlet, thermometer pocket, addition funnel and overhead stirrer. To this, a solution of 2,4,5-trifluorophenyl acetic acid (100 g) in acetonitrile (600 ml) was added slowly over a period of a 30 min at 30° C. and the reaction mixture was stirred at 30° C. for 3 h. This solution was marked as part B.

The Part B solution was added drop-wise to part-A slurry over a period of 2 hour at 30° C. and the mixture was stirred. The progress of the reaction was monitored by TLC (Mobile phase: n-Hexane: ethyl acetate; 50:50). After 12 h, TLC analysis indicated <5% of un-reacted starting material. The reaction mixture was concentrated under reduced pressure at 50-55° C. to get a thick slurry. To this, water (1000 ml) was added and the mixture was cooled to 10-15° C. and conc. HCl (220.6 ml) was added slowly, below 20° C. MTBE (700 ml) was added to the mixture and the mixture was stirred for 30 min. The layers were separated and the aqueous layer was extracted with MTBE (200 ml). The combined organic layers were washed with 7% aqueous NaHCO₃ solution (400 ml) followed by washing with brine (200 ml). The organic layer was dried over sodium sulphate and concentrated under vacuum at 50° C. to get an oil (120 g). The obtained oil was diluted with isopropyl alcohol (400 ml) and cooled to 20° C. To this water (800 ml) was added slowly over a period of 4 hours at 18-20° C. The slurry was then stirred for 4 hours and filtered. The obtained cake was washed with water (100 ml) and then suck dried to obtain free solid of titled product.

Yield: 115 g (83%); m.p.: 37-39° C.; HPLC Purity: ≧95%.

¹H NMR (CDCl₃): 3.64 (s, 3H), 3.75 (s, 2H), 3.85 (s, 2H), 6.88-7.11 (m, 2H).

Mass spectrum: 247[M+1]⁺.

### Step-2

### Preparation of 3(S)-4-(2,4,5-Trifluorophenyl)-3-hydroxybutanoic acid (Formula IV)

A solution of Methyl 4-(2,4,5-trifluorophenyl)-3-oxobutanoate (100 g) in methanol (500 ml) and acetic acid (1.0 ml) was degassed by bubbling nitrogen gas for 15 min. The mixture was transferred to an autoclave and to this (S)—BINAP—RuCl₂ (0.4 g, 0.4 wt %) was added. The reaction mixture was further degassed by nitrogen. The reaction mixture was then hydrogenated (70 psi) at 70° C. for 5-6 h. The progress of the reaction was monitored by TLC till complete conversion of the starting material. After completion of reaction, the methanolic solution of 3-hydroxyester was charged to a RBF equipped with overhead stirrer, thermometer pocket and addition funnel. To the above methanolic solution, water (400 ml) and aqueous solution of NaOH (17.9 g in 100 ml of water) were added at 20-25° C. The mixture was stirred for 1.5 h. At this point TLC analysis indicated complete consumption of starting material. Methanol was removed from the reaction mixture by distillation under vacuum. The mixture was extracted with MTBE (200 ml) and the layers were separated. The aqueous layer was cooled to 10-15° C., acidified using conc. hydrochloric acid and stirred for 2 h. The obtained slurry was filtered to obtain a cake. The cake was washed with water and air-dried to get 3(S)-4-(2,4,5-Trifluorophenyl)-3-hydroxybutanoic acid.

Yield: 81 g (86%); HPLC Purity: ≧90%.

¹H NMR (CDCl₃): 2.49 (s, 2H), 2.80 (s, 2H), 4.22.4.27 (m, 1H), 6.88-7.21 (m, 2H),

Mass spectrum: 234[M+1]⁺.

### Step-3

### Preparation of N-(Benzyloxy)-4(R)-[1-methyl-(2,4,5-trifluorophenyl)]-2-oxoazetidine (Formula V)

A mixture of Step-2 product (80 g), O-benzylhydroxylamine hydrochloride (60 g), LiOH (14.4 g, 1.0 equiv) in THF (200 ml) and water (600 ml) were stirred at 20-22° C. To this, DCC (88 g) was added in one lot. The suspension was stirred for 3 hours. At this point, complete consumption of starting material was observed as indicated by TLC. After completion of reaction, the reaction mixture was diluted with MTBE (650 ml) and the slurry was filtered. The solid was washed with MTBE (160 ml) and the layers were separated. The separated MTBE layer was concentrated to obtain a residue. The obtained residue was stripped with THF until KF of the solution is <0.2%. The residue was diluted with THF and the final volume of the mixture was adjusted to 340 ml and the solution was marked as hydroxamate.

In a separate RBF, to a solution of triphenylphosphine (98.6 g) in THF (400 ml) was slowly added diisopropylazodicarboxylate (DIAD, 74 ml) in such a rate that the temperature did not rise above 10° C. over 30 min. The above solution of hydroxamate was added slowly to the mixture, maintaining the temperature below 10° C. over a period of 30 min. After completion of addition, the reaction mixture was warmed to 20° C. and stirred for 18 h. The TLC analysis indicated complete consumption of the starting material. To this, acetic acid (1.1 g) was added and the mixture was concentrated under vacuum to obtain a residue. The obtained residue was cooled to 25° C. followed by successive addition of methanol (720 ml) and water (56 ml). The obtained solution was cooled to −20° C. to obtain a slurry. The slurry was stirred for 2 h at −20° C. and filtered to get solid which was washed with 10% (v/v) aqueous methanol (2×80 ml ) and dried at room temperature to get N-(Benzyloxy)-4(R)-[1-methyl-(2,4,5-trifluorophenyl)-2-oxoazetidine.

Yield: 83 g (76%); m.p.: 70-72° C.; HPLC Purity: ≧95%, Chiral HPLC: ≧99%.

¹H NMR (CDCl₃): 2.35 (d, 2H), 2.36-2.91 (m, 3H), 3.62-3.65 (m, 1H), 6.88-6.97 (m, 2H),7.29-7.43 (m, 5H).

Mass spectrum: 322[M+H]⁺

### Step 4

### Preparation of (3R)-3-[(benzyloxy)amino]-1-[3-(trifluoromethyl)-5H, 6H,7H,8H [1,2,4]triazolo[4,3-a]pyrazin-7-yl]-4-(2,4,5-trifluorophenyl)butan-1-one (Formula VII)

N-(Benzyloxy)-4(R)-[1-methyl-(2,4,5-trifluorophenyl)]-2-xoazetidine(80 g), THF (240 ml) and water (240 ml) were charged to a four neck RBF equipped with an overhead stirrer and a thermometer pocket followed by addition of lithium hydroxide (14.4 g) at 20-25° C. over a period of 20 min. The resulting mixture was stirred at room temperature for 2 hours and progress of the reaction was monitored by TLC (complete conversion of the starting material). The pH of the reaction mixture was adjusted to about 3 using methanesulfonic acid (22.3 ml) maintaining temperature below 20° C. The suspension was extracted with MTBE (600 ml) and the separated MTBE layer was concentrated to obtain a thick oil. The obtained oil was diluted with acetonitrile (900 ml) and triazole HCl of formula (VI) (65.5 g) was added to the obtained solution. The mixture was cooled to 0-5° C. and N-Methyl morpholine (23.2 g, NMM) was added to it. To this, EDC-HCl (66.2 g) was charged and the mixture was stirred for 3 h at 0-5° C. After completion of reaction, the mixture was diluted with water (325 ml) and MTBE (650 ml). The layers were separated and the organic layer was washed with 10% aqueous KHCO₃ solution (320 ml) followed by brine (320 ml). The organic layer was concentrated to obtain a thick oil. The oil was diluted with ethanol and the ethanolic solution (160 g) was taken up for hydrogenation.

Yield: 110 g (86%); HPLC Purity: ≧95%.

¹H NMR (DMSO d₆): 2.35 (d, 2H), 2.36-2.91 (m, 3H), 3.62-3.65 (m, 1H), 6.88-6.97 (m, 2H),7.29-7.43 (m, 5H).

Mass spectrum: 514[M+H]⁺

### Step-5

### Preparation of 3(R)-3-Amino-1-[3-(trifluoromethyl)-5H,6H,7H,8H [1,2,4]triazolo[4,3-a]pyrazin-7-yl]-4-(2,4,5-trifluorophenyl)butan-1-one phosphate (Sitagliptin phosphate)

### Procedure A

Ethanolic solution of Step 4 product {3(R)-3-[(benzyloxy)amino]-1-[3-(trifluoromethyl)-5H, 6H,7H,8H[1,2,4]triazolo[4,3-a]pyrazin-7-yl]-4-(2,4,5-trifluoro phenyl) butan-1-one}(100 g), ethanol (500 ml), water (43 ml), 10% Pd/C (20 g, 50% wet) and conc. HCl (14 ml) were charged to a 2 L autoclave. The autoclave was pressurized to 40 psi and the mixture was heated at 30° C. for 12-14 hours. The solution was filtered through a hyflow bed (50 g) and the resulting solution was concentrated to get a residue. The obtained residue was diluted with water (50 ml) and pH of the solution was adjusted to 13 using NaOH (8.2 g) at a temperature between 12-14° C. The suspension was extracted with MTBE (500 ml) and the separated organic layer was concentrated to get Sitagliptin as an oil (58 g). The obtained crude Sitagliptin base (58 g), ethanol (300 ml) and water (50 ml) were charged to a 1 L flask equipped with an overhead stirrer, water bath and thermometer pocket. The solution was heated to 45° C. followed by addition of 85% aqueous phosphoric acid solution(16.7 g). The mixture was heated to 75° C. to obtain a thick white precipitate. The obtained slurry was cooled to 65-68° C. for 2 h and further cooled to 25° C. followed by stirring the mixture for 12 h. After completion of reaction, the obtained slurry was filtered to obtain a solid, which was washed with ethanol (100 ml) and air-dried.

Yield: 52 g (66%); m.p.: 214-216° C.; HPLC Purity: >99.7%.

¹H NMR (400 MHz, D₂O): 7.08-7.26(m, 2H), 4.89-4.90 (m, 2H), 3.40-4.14(m, 5H), 2.49-2.68 (m, 4H).

Mass spectrum: 408[M+1]⁺

### Procedure B

Ethanolic solution of Step 4 product {3(R)-3-[(benzyloxy)amino]-1-[3-((trifluoromethyl)-5H, 6H,7H,8H[1,2,4]triazolo[4,3-a]pyrazin-7-yl]-4-(2,4,5-trifluoro phenyl)butan-1-one}(100 g), ethanol (500 ml), water (50 ml), 10% Pd/C (10 g, 50% wet) and benzylchloride (20 ml) were charged to an autoclave. The autoclave was pressurized with hydrogen to 40 psi and the mixture was stirred at 40□C for 4-5 hours. The solution was filtered through a hyflow bed (50 g) and the resulting solution was concentrated to obtain an oil. The obtained oil was diluted with water (150 ml) and to this, trithiocyanuric acid (2 g) and charcoal (5 g) were added. The mixture was stirred for 2 hours at 25-30° C. and filtered to obtain a solid. The obtained solid was washed with water (25 ml) and the pH of the filtrate was adjusted to 13 using NaOH (8.2 g in 25 ml of water) at a temperature between 10-15° C. The suspension was extracted with mixture of MTBE (350 ml) and acetonitrile (50 ml). The layers were separated and the aqueous layer was extracted with MTBE (100 ml). The combined organic layers were concentrated to obtain Sitagliptin base (55 g) as an oil. The obtained crude Sitagliptin base (55 g), IPA (105 ml) and water (45 ml) were charged to a 1 L round bottom flask(RBF) equipped with an overhead stirrer, water bath and thermometer pocket. To this, 85% aqueous phosphoric acid solution (16.7 g) was added. The mixture was heated to 75° C. to obtain a clear solution. The obtained solution was cooled to 65-68° C. and stirred for 2 hours. After 2 hours, the solution was cooled to 55-60° C. and to this, a seed of Sitagliptin phosphate monohydrate was added. The slurry was cooled to 25° C. followed by addition of IPA (350 ml) and the slurry was stirred for 12 hours. The obtained slurry was filtered to obtain a solid. The obtained solid was washed with IPA (3×100 ml) and the solid was air-dried to obtain the titled product.

Yield: 100 g (63%); HPLC Purity: >99.7%.

¹H NMR (400 MHz, D₂O): 7.08-7.26(m, 2H), 4.89-4.90 (m, 2H), 3.40-4.14(m, 5H), 2.49-2.68 (m, 4H).

Mass spectrum: 408[M+1]⁺

### Step-6

### Purification of Sitagliptin Phosphate Monohydrate

Crude Sitagliptin phosphate monohydrate of step-6(50 gm), IPA(105 ml ) and water(45 ml) were charged to a round bottom flask equipped with an overhead stirrer, water bath and thermometer pocket to obtain a slurry. The slurry was heated to 75° C. to obtain a solution. The solution was cooled to 55° C. to 60° C. and to this, a seed of Sitagliptin phosphate monohydrate was added. The solution was further cooled at 25° C. and to this IPA (350 ml ) was added over a period of 1 hour and the obtained slurry was stirred for 12 hours. The obtained slurry was filtered to obtain a solid, which was washed with IPA(2×50m1). The product was dried at 45° C. to 50° C. for 12 hours to obtain pure Sitagliptin phosphate monohydrate.

### Example 2

The particle size distribution (PSD) of Sitagliptin was carried out using dry mode (Scirocco) method and following conditions:

Instrument: Malvern mastersizer 2000

Vibration: 70%

Dispersive air pressure: 3.0 bar

Particle refractive index: 1.52

Absorption: 0.1

Background: 10 seconds

Sample measurement time: 10 seconds

Measurement cycle: 3 measurement per aliquot

Delay: 10 seconds

Slit width: 3 mm

Obscuration: 1 to 5%

### Example 3

### Preparation of 3(R)-3-Amino-1-[3-(trifluoromethyl)-5H,6H,7H,8H[1,2,4 ]triazolo[4,3-a]pyrazin-7-yl]-4-(2,5-difluorophenyl)butan-1-one phosphate (Impurity A)

This compound was prepared using a procedure as given in Example 1 using 2,5-difluorophenylacetic acid in place of 2,4,5-trifluorophenyl acetic acid.

¹H NMR (400 MHz, D₂O): 6.95-7.0 (m, 3H), 4.66-4.86 (m, 2H), 3.86-4.18 (m, 5H), 2.80-3.05 (m, H); Mass spectrum: 390[M+1]⁺

### Example 4

### Preparation of 3(R)-3-Amino-1-[3-(trifluoromethyl)-5H,6H,7H,8H[1,2,4]triazolo[4,3-a]pyrazin-7-yl]-4-(2,4-difluorophenyl)butan-1-one phosphate (Impurity B)

This compound was prepared using a procedure as given in Example 1 using 2,4-difluorophenylacetic acid in place of 2,4,5-trifluorophenyl acetic acid.

¹H NMR (400 MHz, D₂O): 6.74-7.21 (m, 3H), 4.67-4.79 (m, 2H), 3.85-4.11 (m,5H), 2.74-3.08 (m, 4H); Mass spectrum: 390[M+1]⁺

### Example 5

### Preparation of 3(R)-3-Amino-1-[3-(trifluoromethyl)-5H,6H,7H,8H[1,2,4 ]triazolo[4,3-a]pyrazin-7-yl]-4-(3,4-difluorophenyl)butan-1-one phosphate (Impurity C)

This compound was prepared in a similar procedure as given in Example 1 using 3,4-difluorophenylacetic acid in place of 2,4,5-trifluorophenyl acetic acid.

¹H NMR (400 MHz, D₂O): 6.97-7.14 (m, 3H), 4.66-4.84 (m, 2H), 3.86-4.19 (m, 5H), 2.77-2.98 (m, 4H); Mass spectrum: 390[M+1]⁺

### Example 6

### Preparation of Sitagliptin

Sitagliptin phosphate (20 g) was suspended in water (100 ml) and the obtained suspension was cooled to 0-5° C. Sodium hydroxide (2.3 g) was added to the cooled suspension and the slurry was stirred for 1 h at 0-5° C. The obtained solid was filtered, washed with water (40 ml) and dried at 30° C. for 12 h to obtain 15 g of Sitagliptin. Yield: 95%

### Example 7

### Preparation of Sitagliptin Phosphate

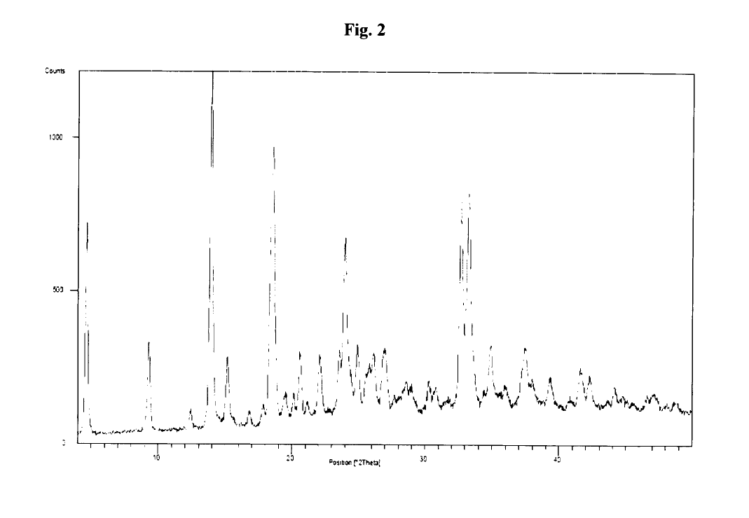

0.24 g of phosphoric acid (99%) was dissolved in 20 ml of IPA at 30° C. to obtain a solution. Sitagliptin base (1.0 g) was added to obtained solution at 30° C. and the mixture was stirred for 16 h at the same temperature. The mixture was then cooled to about 8° C. and stirred for several hours. The solid obtained was filtered and dried at 30-35° C. under vacuum to obtain 0.75 g of Sitagliptin phosphate characterized by X-ray diffraction pattern as shown in FIG. 2 having peaks expressed as 2-theta values of about 4.73, 9.39, 12.42, 13.93, 15.16, 16.80, 18.38, 18.60, 19.55, 20.11, 20.58, 22.10, 23.55, 23.99, 24.93, 26.17, 26.97, 30.28, 30.80, 32.66, 33.23, 34.89, 37.51, 39.29, 41.59, 42.28 and 44.13 degrees.

### Example 8

### Preparation of Sitagliptin Phosphate

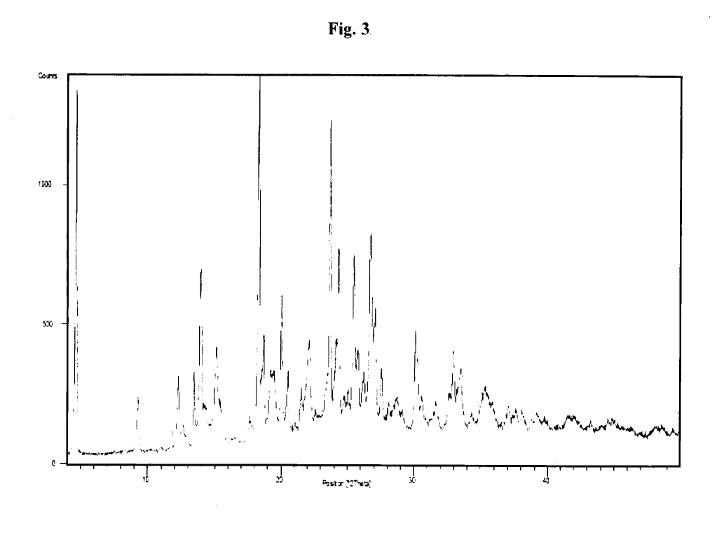

Sitagliptin free base (58 g), ethanol (300 ml) and water (50 ml) were charged to a 1 L round bottom flask and the solution was heated to 45° C. 85% aqueous phosphoric acid (16.7 g) was added to the solution and the reaction mixture was heated to 75° C. A thick white precipitate was formed. The obtained slurry was cooled to 65-68° C. and was held at the same temperature for 2 hours. The slurry was cooled to 25° C. gradually, stirred for 12 h and obtained solid of Sitagliptin phosphate was filtered, washed with ethanol (100 ml) and dried to obtain Sitagliptin phosphate characterized by X-ray diffraction pattern as shown in FIG. 3 having peaks expressed as 2-theta values of about 4.68, 9.34, 11.78, 12.28, 13.46, 13.99, 15.13, 17.64, 18.31, 18.67, 19.47, 20.00, 20.49, 21.49, 22.14, 23.67, 24.31, 25.46, 25.81, 26.24, 26.67, 27.09, 27.56, 28.72, 30.09, 31.64, 32.95, 33.51 and 37.04 degrees.

### Example 9

### Preparation of Sitagliptin Phosphate Monohydrate

Sitagliptin (58 g), IPA (105 ml) and water (45 ml) were charged to a 1 L round bottom flask and the solution was stirred for 15 min. To this, 85% aqueous phosphoric acid (16.5 g) was added and the mixture was heated to 75° C. to get a clear solution. The clear solution was cooled to 65-68° C. and held at that temperature for 2 hours. The slurry was cooled to 55° C. gradually and seeded with Sitagliptin phosphate monohydrate. The slurry was cooled to 25° C., and to this IPA (350 ml) was added and stirred for 12 hours. The obtained solid was filtered, washed with IPA (2×100 ml) and dried to obtain 50 g of Sitagliptin phosphate monohydrate.

### Example 10

### Preparation of Sitagliptin HCl Form III

Sitagliptin base (10 g) was suspended in 150 ml of IPA. 3 ml of concentrated hydrochloric acid (36%) was added to the obtained suspension at 30° C. The solution was stirred for 3 hours. The obtained solid was filtered and dried at 60° C. to obtain 8.5 g Sitagliptin HCl Form III.

### Example 11

### Preparation of Sitagliptin HCl Form III

Sitagliptin base (10 g) was suspended in 100 ml of IPA. The suspension was stirred for 30 min at 30° C. 3 ml of aqueous hydrochloric acid (35%) was added to the suspension. 50 ml of IPA was added to the obtained thick suspension and the suspension was stirred for 24 hours. The obtained solid was filtered and dried at 55-65° C. to obtain 6.8 g Sitagliptin HCl Form III.

### Example 12

### Preparation of Sitagliptin HCl Form IV

0.5 g of Sitagliptin HCl Form III was dissolved in 5 ml of acetone at reflux. The hot solution was filtered and poured in 20 ml of diisopropylether at 30° C. The obtained sticky mass was stirred at same temperature for 5 hours. 40 ml of DIPE was again added to the obtained mass and stirred for 48 hours. The obtained solid was filtered and dried at 55° C. to obtain 0.35 g of Sitagliptin HCl Form IV.

### Example 13

### Preparation of Sitagliptin HCl Form V

1 g of Sitagliptin base was suspended in 20 ml of diethyl ether at 25° C. Dry HCl gas was passed through the obtained suspension to attain pH 3-4. The suspension was stirred for 30 min. The separated solid was isolated and filtered to obtain 0.8 g Sitagliptin HCl Form V.

### Example 14

### Sitagliptin Esylate Form I

0.27 g of ethane sulfonic acid was dissolved in 20 ml of isopropylalcohol (IPA) and 1 g of Sitagliptin base was added to the obtained solution. The solution was stirred at 30° C. for 2 hours. The separated solid was filtered, washed with IPA and dried at 60° C. to obtain 0.52 g of Sitagliptin esylate Form I.

### Example 15

### Sitagliptin Esylate Form II

1 g of Sitagliptin esylate Form I was dissolved in 10 ml of methanol at 60° C. The hot solution was filtered and the filtrate was added over 40 ml diisopropylether at 30° C. The obtained solution was stirred for several hours at same temperature. The separated solid was filtered to obtain 0.75 g of Sitagliptin esylate Form II.

### Example 16

### Sitagliptin Esylate Form III

0.5 g of Sitagliptin esylate was suspended in 20 ml of ethyl acetate. The suspension was heated to reflux temperature followed by addition of 5 ml of ethanol to get clear solution. The hot solution was filtered to remove suspended particles. The filtrate was cooled to 25° C. and stirred for 3-4 hours. The separated solid was filtered and dried at 60° C. to obtain Sitagliptin esylate Form III.

1. A process for preparation of Sitagliptin or pharmaceutically acceptable salts thereof comprising the steps of,
a) hydrogenating methyl 4-(2,4,5-trifluorophenyl)-3-oxobutanoate in presence of less than about 0.5% w/w of (S)—BINAP—RuCl₂ with respect to methyl 4-(2,4,5-trifluorophenyl)-3-oxobutanoate to obtain 3(S)-4-(2,4,5-trifluorophenyl)-3-hydroxybutanoic acid; and
b) converting said 3(S)-4-(2,4,5-trifluorophenyl)-3-hydroxybutanoic acid to Sitagliptin or pharmaceutically acceptable salts thereof.

2. The process as claimed in claim 1, wherein said hydrogenation in step a) is carried out at a temperature of about 60° C. to 80° C. and pressure of about 60 to 80 psi for about 5 to 6 hours in presence of organic acid selected from acetic acid, formic acid, citric acid, lactic acid or tartaric acid.

3. The process as claimed in claim 1, wherein said conversion in step b) comprises the steps of,
a) treating said 3(S)-4-(2,4,5-trifluorophenyl)-3-hydroxybutanoic acid with protected hydroxylamine in presence of a coupling agent followed by cyclocondensation in presence of phosphine ligand and azodicarboxylate to obtain N-Benzyloxy-4(R)[1-methyl-(2,4,5-trifluorophenyl)]-2-oxoazetidine;
b) subjecting said N-Benzyloxy-4(R)-[1-methyl-(2,4,5-trifluorophenyl)]-2-oxoazetidine to ring opening followed by treatment with 3-(trifluoromethyl)-5H,6H,7H, 8H-[1,2,4]triazolo[4,3-a]pyrazine or its salt in presence of a coupling agent to obtain 3(R)-3-[(benzyloxy)amino]-1-[3-(trifluoromethyl)-5H,6H,7H,8H-[1,2,4]triazolo[4,3-a]pyrazin-7-yl]-4-(2,4,5-trifluorophenyl)butan-1-one;
c) subjecting said 3(R)-3-[(benzyloxy)amino]-1-[3-(trifluoromethyl)-5H,6H,7H, 8H-[1,2,4]triazolo[4,3-a]pyrazin-7-yl]-4-(2,4,5-trifluorophenyl)butan-1-one to debenzyloxylation in presence of a suitable catalyst and additive to obtain Sitagliptin;
d) optionally converting said Sitagliptin into its pharmaceutically acceptable salts thereof, which is optionally purified.

4. The process as claimed in claim 3, wherein said coupling agent is selected from N,N′-dicyclohexylcarbodiimide, 1-ethyl-3-(3-dimethyl amino propyl) carbodiimide or N,N′-diisopropylcarbodiimide; said phosphine ligand is selected from triphenylphosphine, tri(o-tolyl)phosphine, tributylphosphine or trioctylphosphine; and said azodicarboxylate is selected from diisopropylazodicarboxylate (DIAD), diethylazodicarboxylate (DEAD) or dibenzylazodicarboxylate.

5. The process as claimed in claim 3, wherein said debenzyloxylation in step c) is carried out using hydrogen gas at a temperature of about 30 to 50° C. and pressure of about 30 to 50 psi for about 3 to 6 hours; said suitable catalyst is selected from palladium, platinum, rhodium or nickel on supports such as carbon, silica or alumina, oxides thereof or salts thereof; and said additive is selected from benzyl chloride, benzyl bromide, benzyl iodide or substituted derivatives thereof.

6. A process for preparation of Sitagliptin or pharmaceutically acceptable salts thereof comprising the steps of,
a) debenzyloxylation of 3(R)-3-[(benzyloxy)amino]-1-[3-(trifluoromethyl)-5H, 6H,7H,8H-[1,2,4]triazolo[4,3-a]pyrazin-7-yl]-4-(2,4,5-trifluoro phenyl)butan-1-one using hydrogen gas in presence of a catalyst and an additive to obtain a reaction mixture, wherein said additive is selected from benzyl chloride, benzyl bromide, benzyl iodide or substituted derivatives thereof;
b) isolating Sitagliptin from said reaction mixture.

7. The process as claimed in claim 6, wherein said debenzyloxylation is carried out at a temperature of about 40° C. and pressure of about 40 psi for about 4 to 5 hours; and said catalyst is selected from palladium, platinum, rhodium or nickel on supports such as carbon, silica or alumina, oxides thereof or salts thereof.

8. The process as claimed in claim 6, wherein said debenzyloxylation of 3(R)-3-[(benzyloxy)amino]-1-[3-(trifluoromethyl)-5H,6H,7H,8H-[1,2,4]triazolo[4,3-a]pyrazin-7-yl]-4-(2,4,5-trifluoro phenyl)butan-1-one is carried out in presence of palladium on carbon support and additive selected from benzyl chloride, benzyl bromide, benzyl iodide or substituted derivatives thereof to obtain a reaction mixture.

9. The process as claimed in claim 8, wherein said reaction mixture is further treated with trithiocyanuric acid to reduce the palladium content prior to isolation of Sitagliptin.

10. The process as claimed in claim 3, wherein said conversion of Sitagliptin to pharmaceutically acceptable salts thereof comprises the steps of,
a) dissolving Sitagliptin in a suitable solvent selected from methanol, ethanol, n-propanol, isopropanol, butanol, water, acetone, 2-butanone, diethyl ketone, diethyl ether, diisopropyl ether, ethyl acetate, methyl acetate, propyl acetate, butyl acetate or mixture thereof to obtain a solution;
b) adding salt forming agent selected from phosphoric acid, HCl, SOCl₂, NH₄Cl, HBr, methane sulfonic acid or ethane sulfonic acid to said solution to obtain a mixture;
c) optionally adding an antisolvent to said mixture; and
d) isolating pharmaceutically acceptable salts of Sitagliptin from said mixture.

11. The process as claimed in claim 10, wherein said salt forming agent is phosphoric acid; said pharmaceutically acceptable salt is Sitagliptin phosphate;
and said suitable solvent is selected from ethanol, isopropanol, water or mixture thereof.

12. The process as claimed in claim 5 wherein said Sitagliptin or pharmaceutically acceptable salt thereof is substantially free of compound selected from group consisting of 3(R)-3-Amino-1-[3-(trifluoromethyl)-5H, 6H,7H,8H-[1,2,4]triazolo[4,3-a]pyrazin-7-yl]-4-(2,5 -difluorophenyl) butan-1-one; 3(R)-3 -Amino-1-[3 -(trifluoromethyl)-5H,6H,7H,8H-[1,2,4 ]triazolo[4,3 -a]pyrazin-7-yl]-4-(2,4-difluorophenyl)butan-1-one and 3(R)-3 -Amino-1-[3-(trifluoromethyl)-5H, 6H,7H,8H-[1,2,4]triazolo[4,3-a]pyrazin-7-yl]-4-(3,4-difluorophenyl)butan-1-one.

13. The process as claimed in claim 12 further comprising the use of compound selected from the group consisting of 3(R)-3-Amino-1-[3-(trifluoromethyl)-5H, 6H,7H,8H-[1,2,4]triazolo[4,3-a]pyrazin-7-yl]-4-(2,5-difluorophenyl)butan-1-one; 3(R)-3-Amino-1-[3-(trifluoromethyl)-5H,6H,7H, 8H-[1,2,4]triazolo[4,3-a]pyrazin-7-yl]-4-(2,4-difluorophenyl)butan-1-one and 3(R)-3-Amino-1-[3-(trifluoromethyl)-5H,6H,7H,8H-[1,2,4]triazolo[4,3-a]pyrazin-7-yl]-4-(3,4-difluorophenyl)butan-1-one as a reference marker and/or reference standard in determining the purity of a sample of Sitagliptin or a pharmaceutical dosage form comprising Sitagliptin.

14. (canceled)

15. The process as claimed in claim 10, wherein said salt forming agent is HCl; said pharmaceutically acceptable salt is Sitagliptin HCl; and said suitable solvent is selected from acetone, 2-butanone, diethyl ketone, diethyl ether, isopropanol or mixture thereof.

16. The process as claimed in claim 15, wherein said Sitagliptin HCl is characterized by X-ray diffraction pattern having peaks at 2-theta values of about 6.50, 7.96, 13.69, 16.01, 17.97, 18.61, 19.72, 20.26, 22.56, 24.63, 25.35, 25.60, 26.98, 29.31 and 31.54 degrees; or X-ray diffraction pattern having peaks at 2-theta values of about 5.18, 10.36, 12.72, 15.59, 16.06, 16.64, 17.27, 17.54, 19.85, 22.54, 23.62, 23.86, 24.22, 25.72, 26.25, 26.94, 28.09, 28.33 and 28.64 degrees; or X-ray diffraction pattern having peaks at 2-theta values of about 7.30, 7.89, 11.80, 15.77, 16.48, 17.86, 18.09, 20.30, 20.51, 20.88, 21.45, 24.05, 24.71, 25.16 and 25.50 degrees.

17. The process as claimed in claim 10, wherein said salt forming agent is ethane sulfonic acid; said pharmaceutically acceptable salt is Sitagliptin esylate; and said suitable solvent is selected from methanol, ethanol, n-propanol, isopropanol, butanol, ethyl acetate, methyl acetate, propyl acetate, butyl acetate or mixture thereof; and said anti solvent is selected from diisopropyl ether, diethyl ether, methyl tert-butyl ether, THF or 1,4-dioxane.

18. The process as claimed in claim 17, wherein said Sitagliptin esylate is characterized by X-ray diffraction pattern having peaks at 2-theta values of about 6.83, 10.66, 12.10, 13.30, 13.67, 15.09, 15.65, 17.22, 18.41, 20.55, 21.49, 22.49, 24.36, 25.71, 27.34, 27.84 and 28.34 degrees; or X-ray diffraction pattern having peaks at 2-theta values of about 5.27, 10.63, 14.92, 15.51, 16.85, 19.26, 21.32, 22.48, 23.35, 24.17, 24.36, 25.23, 25.53 and 32.20 degrees; or X-ray diffraction pattern having peaks at 2-theta values of about 6.86, 13.73, 16.47, 20.60, 23.04, 26.89, 27.83, 33.82 and 34.66 degrees.

19. (canceled)
